SEQUENCE LISTING (137 sequences)

SEQ ID NO: 1 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 2 - protein, 21 aa, Homo sapiens
MEVGWYRPPFSRVVHLYRNGK

SEQ ID NO: 3 - protein, 21 aa, Homo sapiens
MEVGWYRSPFSRVVHLYRNGK

SEQ ID NO: 4 - protein, 20 aa, Homo sapiens
EVGWYRSPFSRVVHLYRNGK

SEQ ID NO: 5 - protein, 20 aa, Homo sapiens
GQFRVIGPRHPIRALVGDEV

SEQ ID NO: 6 - protein, 15 aa, Homo sapiens
DEGGYTCFFRDHSYQ

SEQ ID NO: 7 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 8 - protein, 13 aa, Homo sapiens
HCLGKWLGHPDKF

SEQ ID NO: 9 - protein, 14 aa, Homo sapiens
NTWTTSQSIAFPSK

SEQ ID NO: 10 - protein, 52 aa, Homo sapiens
HCLGKWLGHPDKFVGITYALTVVWLLVFACSAVPVYIYFNTWTTCQSIAFPS

SEQ ID NO: 11 - protein, 16 aa, Homo sapiens
HCLGKWLGHPDKFVGI

SEQ ID NO: 12 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 13 - protein, 20 aa, Homo sapiens
KYLATASTMDHARHGFLPRH

SEQ ID NO: 14 - protein, 25 aa, Homo sapiens
AQGTLSKIFKLGGRDSRSGSPMARR

SEQ ID NO: 15 - protein, 21 aa, Homo sapiens
VHFFKNIVTPRTPPPSQGKGR

SEQ ID NO: 16 - protein, 19 aa, Homo sapiens
LSRFSWGAEGQRPGFGYGG

SEQ ID NO: 17 - protein, 17 aa, Homo sapiens
ENPVVHFFKNIVTPRTP

SEQ ID NO: 18 - protein, 19 aa, Homo sapiens
LHRSGVLIIHHLQEDYRTY

SEQ ID NO: 19 - protein, 9 aa, Homo sapiens
KYNKANVFL

SEQ ID NO: 20 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 21 - protein, 58 aa, Homo sapiens
SHLVEALYLVCGERGFFYTPKTRREAEDLQVGQVELGGGPGAGSLQPLALEGSLQSRG

SEQ ID NO: 22 - protein, 15 aa, Homo sapiens
SHLVEALYLVCGERG

SEQ ID NO: 23 - protein, 21 aa, Homo sapiens
GIVEQCCTSICSLYQLENYCN

SEQ ID NO: 24 - protein, 30 aa, Homo sapiens
FVNQHLCGSHLVEALYLVCGERGFFYTPKT

SEQ ID NO: 25 - protein, 31 aa, Homo sapiens
EAEDLQVGQVELGGGPGAGSLQPLALEGSLQ

SEQ ID NO: 26 - protein, 18 aa, Homo sapiens
GAGSLQPLALEGSLQKRG

SEQ ID NO: 27 - protein, 15 aa, Homo sapiens
SLQPLALEGSLQSRG

SEQ ID NO: 28 - protein, 7 aa, Triticum sp.
PQPEQPF

SEQ ID NO: 29 - protein, 7 aa, Triticum sp.
PQPELPY

SEQ ID NO: 30 - protein, 5 aa, Triticum sp.
EQEQP

SEQ ID NO: 31 - protein, 9 aa, Triticum sp.
EGSFQPSQE

SEQ ID NO: 32 - protein, 33 aa, triticum sp.
LQLQPFPQPELPYPQPQLPYPQPELPYPQPQPF

SEQ ID NO: 33 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 34 - protein, 34 aa, Homo sapiens
RPSTSRSLYASSPGGVYATRSSAVRLRSSVPGVR

SEQ ID NO: 35 - protein, 22 aa, Homo sapiens
PGGVYATRSSAVRLRSSVPGVR

SEQ ID NO: 36 - protein, 466 aa, synthetic construct
  SITE 64: note = citrulline
  SITE 69: note = citrulline
  SITE 71: note = citrulline
  SITE 78: note = citrulline
MSTRSVSSSSYRRMFGGPGTASRPSSSRSYVTTSTRTYSLGSALRPSTSRSLYASSPGGVYATXSSAVXLXSSVPGVXLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 37 - protein, 34 aa, synthetic construct
  SITE 6: note = citrulline
  SITE 20: note = citrulline
  SITE 25: note = citrulline
RPSTSXSLYASSPGGVYATXSSAVXLRSSVPGVR

SEQ ID NO: 38 - protein, 34 aa, synthetic construct
  SITE 6: note = citrulline
  SITE 20: note = citrulline
RPSTSXSLYASSPGGVYATXSSAVRLRSSVPGVR

SEQ ID NO: 39 - protein, 34 aa, synthetic construct
  SITE 6: note = citrulline
  SITE 25: note = citrulline
RPSTSXSLYASSPGGVYATRSSAVXLRSSVPGVR

SEQ ID NO: 40 - protein, 34 aa, synthetic construct
  SITE 20: note = citrulline
  SITE 25: note = citrulline
RPSTSRSLYASSPGGVYATXSSAVXLRSSVPGVR

SEQ ID NO: 41 - protein, 34 aa, synthetic construct
  SITE 6: note = citrulline
RPSTSXSLYASSPGGVYATRSSAVRLRSSVPGVR

SEQ ID NO: 42 - protein, 34 aa, synthetic construct
  SITE 20: note = citrulline
RPSTSRSLYASSPGGVYATXSSAVRLRSSVPGVR

SEQ ID NO: 43 - protein, 34 aa, synthetic construct
  SITE 25: note = citrulline
RPSTSRSLYASSPGGVYATRSSAVXLRSSVPGVR

SEQ ID NO: 44 - protein, 22 aa, synthetic construct
  SITE 8: note = citrulline
  SITE 13: note = citrulline
  SITE 22: note = citrulline
PGGVYATXSSAVXLRSSVPGVX

SEQ ID NO: 45 - protein, 22 aa, synthetic construct
  SITE 8: note = citrulline
  SITE 22: note = citrulline
PGGVYATXSSAVRLRSSVPGVX

SEQ ID NO: 46 - protein, 22 aa, synthetic construct
  SITE 8: note = citrulline
  SITE 13: note = citrulline
PGGVYATXSSAVXLRSSVPGVR

SEQ ID NO: 47 - protein, 22 aa, synthetic construct
  SITE 13: note = citrulline
  SITE 22: note = citrulline
PGGVYATRSSAVXLRSSVPGVX

SEQ ID NO: 48 - protein, 22 aa, synthetic construct
  SITE 8: note = citrulline
PGGVYATXSSAVRLRSSVPGVR

SEQ ID NO: 49 - protein, 22 aa, synthetic construct
  SITE 13: note = citrulline
PGGVYATRSSAVXLRSSVPGVR

SEQ ID NO: 50 - protein, 22 aa, synthetic construct
  SITE 22: note = citrulline
PGGVYATRSSAVRLRSSVPGVX

SEQ ID NO: 51 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 52 - protein, 151 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYH

SEQ ID NO: 53 - protein, 457 aa, Homo sapiens
MEPWPLLLLFSLCSAGLVLGSEHETRLVAKLFKDYSSVVRPVEDHRQVVEVTVGLQLIQLINVDEVNQIVTTNVRLKQQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRLPLYFIVNVIIPCLLFSFLTGLVFYLPTDSGEKMTLSISVLLSLTVFLLVIVELIPSTSSAVPLIGKYMLFTMVFVIASIIITVIVINTHHRSPSTHVMPNWVRKVFIDTIPNIMFFSTMKRPSREKQDKKIFTEDIDISDISGKPGPPPMGFHSPLIKHPEVKSAIEGIKYIAETMKSDQESNNAAAEWKYVAMVMDHILLGVFMLVCIIGTLAVFAGRLIELNQQG

SEQ ID NO: 54 - protein, 235 aa, Homo sapiens
SEHETRLVAKLFKDYSSVVRPVEDHRQVVEVTVGLQLIQLINVDEVNQIVTTNVRLKQGDMVDLPRPSCVTLGVPLFSHLQNEQWVDYNLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPESDQPDLSNFMESGEWVIKESRGWKHSVTYSCCPDTPYLDITYHFVMQRL

SEQ ID NO: 55 - protein, 99 aa, Homo sapiens
NLKWNPDDYGGVKKIHIPSEKIWRPDLVLYNNADGDFAIVKFTKVLLQYTGHITWTPPAIFKSYCEIIVTHFPFDEQNCSMKLGTWTYDGSVVAINPES

SEQ ID NO: 56 - protein, 55 aa, Homo sapiens
LLQYTGHITWTPPAIFKSYCEIIVTHFPFDEQNCSMKLGTWTYDGSVVAINPESD

SEQ ID NO: 57 - protein, 20 aa, Homo sapiens
MKLGTWTYDGSVVAINPESD

SEQ ID NO: 58 - protein, 470 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEVVTESQKEQRSELDKSSAHSY

SEQ ID NO: 59 - protein, 40 aa, Homo sapiens
DSHSRKTVLIKTIETRNGEVVTESQKEQRSELDKSSAHSY

SEQ ID NO: 60 - protein, 41 aa, Homo sapiens
DSHSKKMVLIRTIETRDGEKVVTESQKEQHSDLDKSSIHSY

SEQ ID NO: 61 - protein, 323 aa, Homo sapiens
MSDRPTARRWGKCGPLCTRENIMVAFKGVWTQAFWKAVTAEFLAMLIFVLLSLGSTINWGGTEKPLPVDMVLISLCFGLSIATMVQCFGHISGGHINPAVTVAMVCTRKISIAKSVFYIAAQCLGAIIGAGILYLVTPPSVVGGLGVTMVHGNLTAGHGLLVELIITFQLVFTIFASCDSKRTDVTGSIALAIGFSVAIGHLFAINYTGASMNPARSFGPAVIMGNWENHWIYWVGPIIGAVLAGGLYEYVFCPDVEFKRRFKEAFSKAAQQTKGSYMEVEDNRSQVETDDLILKPGVVHVIDVDRGEEKKGKDQSGEVLSSV

SEQ ID NO: 62 - protein, 21 aa, Homo sapiens
LVTPPSVVGGLGVTMVHGNLT

SEQ ID NO: 63 - protein, 25 aa, Homo sapiens
LVTPPSVVGGLGVTMVHGNLTAGHG

SEQ ID NO: 64 - protein, 42 aa, Homo sapiens
LVTPPSVVGGLGVTMVHGNLTAGHGLLVELIITFQLVFTIFA

SEQ ID NO: 65 - protein, 40 aa, Homo sapiens
VFYIAAQCLGAIIGAGILYLVTPPSVVGGLGVTMVHGNLT

SEQ ID NO: 66 - protein, 1427 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 67 - protein, 130 aa, Homo sapiens
SPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQA

SEQ ID NO: 68 - protein, 236 aa, Homo sapiens
CGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 69 - protein, 107 aa, Homo sapiens
CGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRE

SEQ ID NO: 70 - protein, 119 aa, Homo sapiens
EIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 71 - protein, 16 aa, Homo sapiens
IGAELLRDPSLGAQFR

SEQ ID NO: 72 - protein, 19 aa, Homo sapiens
GPSGHVMASDGAAPRAGLA

SEQ ID NO: 73 - protein, 19 aa, Homo sapiens
SPGGASFYHWGAAVPHSQG

SEQ ID NO: 74 - protein, 18 aa, Homo sapiens
KRGDSFLDGTRCMPSGPR

SEQ ID NO: 75 - protein, 13 aa, Homo sapiens
WVNYSCLDQARKE

SEQ ID NO: 76 - protein, 19 aa, Homo sapiens
ARKELVETVQCQGSQQPPA

SEQ ID NO: 77 - protein, 15 aa, Homo sapiens
GSLLKTLPPARCRAG

SEQ ID NO: 78 - protein, 15 aa, Homo sapiens
VVRRILYCARAHGED

SEQ ID NO: 79 - protein, 27 aa, Homo sapiens
RAHGEDDGEEILLDTQCQGLPRPEPQE

SEQ ID NO: 80 - protein, 18 aa, Homo sapiens
TARRSVACVQLDQGQDVE

SEQ ID NO: 81 - protein, 17 aa, Homo sapiens
RGPGQADCAVAIGRPLG

SEQ ID NO: 82 - protein, 26 aa, Homo sapiens
LDMTFSSKTNTLVVRQRCGRPGGGVL

SEQ ID NO: 83 - protein, 18 aa, Homo sapiens
RPGGGVLLLYGSQLAPET

SEQ ID NO: 84 - protein, 18 aa, Homo sapiens
GCRLFINVAPHARIAIHA

SEQ ID NO: 85 - protein, 17 aa, Homo sapiens
ARIAIHALATNMGAGTE

SEQ ID NO: 86 - protein, 19 aa, Homo sapiens
ASYILIRDTHSLRTTAFHG

SEQ ID NO: 87 - protein, 9 aa, Homo sapiens
ILYCARAHG

SEQ ID NO: 88 - protein, 15 aa, Homo sapiens
GCRLFINVAPHARIA

SEQ ID NO: 89 - protein, 9 aa, Homo sapiens
FINVAPHAR

SEQ ID NO: 90 - protein, 14 aa, Homo sapiens
RRSVACVQLDQGQD

SEQ ID NO: 91 - protein, 9 aa, Homo sapiens
VQLDQGQDV

SEQ ID NO: 92 - protein, 15 aa, Homo sapiens
GPGQADCAVAIGRPL

SEQ ID NO: 93 - protein, 9 aa, Homo sapiens
CAVAIGRPL

SEQ ID NO: 94 - protein, 17 aa, Homo sapiens
RPGGGVLLLYGSQLAPE

SEQ ID NO: 95 - protein, 9 aa, Homo sapiens
LLRYGSQLA

SEQ ID NO: 96 - protein, 16 aa, Homo sapiens
ARIAIHALATNMGAGT

SEQ ID NO: 97 - protein, 15 aa, Homo sapiens
RIAIHALATNMGAGT

SEQ ID NO: 98 - protein, 14 aa, Homo sapiens
RIAIHALATNMGAG

SEQ ID NO: 99 - protein, 14 aa, Homo sapiens
IAIHALATNMGAGT

SEQ ID NO: 100 - protein, 9 aa, Homo sapiens
IHALATNMG

SEQ ID NO: 101 - protein, 15 aa, Homo sapiens
LDMTFSSKTNTLVVR

SEQ ID NO: 102 - protein, 9 aa, Homo sapiens
FSSKTNTLV

SEQ ID NO: 103 - protein, 17 aa, Homo sapiens
ARRSVACCQLDQGQDVE

SEQ ID NO: 104 - protein, 9 aa, Homo sapiens
LKTLPPARC

SEQ ID NO: 105 - protein, 15 aa, Homo sapiens
IGAELLRDPSLGAQF

SEQ ID NO: 106 - protein, 14 aa, Homo sapiens
IGAELLRDPSLGAQ

SEQ ID NO: 107 - protein, 8 aa, Homo sapiens
IGAELLRD

SEQ ID NO: 108 - protein, 9 aa, Homo sapiens
LLRDPSLGA

SEQ ID NO: 109 - protein, 16 aa, Homo sapiens
RRSVACVQLDQGQDVE

SEQ ID NO: 110 - protein, 13 aa, Homo sapiens
RLFINVAPHARIA

SEQ ID NO: 111 - protein, 11 aa, Homo sapiens
RLFINVAPHAR

SEQ ID NO: 112 - protein, 17 aa, Homo sapiens
GPSGHVMASDGAAPRAG

SEQ ID NO: 113 - protein, 9 aa, Homo sapiens
MASDGAAPR

SEQ ID NO: 114 - protein, 26 aa, Homo sapiens
RAHGEDDGEEILLDTQCQGLPRPEPQ

SEQ ID NO: 115 - protein, 16 aa, Homo sapiens
DGEEILLDTQCQGLPR

SEQ ID NO: 116 - protein, 14 aa, Homo sapiens
EEILLDTQCQGLPR

SEQ ID NO: 117 - protein, 9 aa, Homo sapiens
ILLDTQCQG

SEQ ID NO: 118 - protein, 18 aa, Homo sapiens
TNTLVVRQRCGRPGGGVL

SEQ ID NO: 119 - protein, 9 aa, Homo sapiens
LVVRQRCGR

SEQ ID NO: 120 - protein, 15 aa, Homo sapiens
LFINVAPHARIAIHA

SEQ ID NO: 121 - protein, 14 aa, Homo sapiens
FINVAPHARIAIHA

SEQ ID NO: 122 - protein, 13 aa, Homo sapiens
INVAPHARIAIHA

SEQ ID NO: 123 - protein, 18 aa, Homo sapiens
ASYILIRDTHSLRTTAFH

SEQ ID NO: 124 - protein, 17 aa, Homo sapiens
ASYILIRDTHSLRTTAF

SEQ ID NO: 125 - protein, 9 aa, Homo sapiens
LIRDTHSLR

SEQ ID NO: 126 - protein, 16 aa, Homo sapiens
GDSFLDGTRCMPSGPR

SEQ ID NO: 127 - protein, 9 aa, Homo sapiens
LDGTRCMPS

SEQ ID NO: 128 - protein, 9 aa, Homo sapiens
QADCAVAIG

SEQ ID NO: 129 - protein, 9 aa, Homo sapiens
LRDPSLGAQ

SEQ ID NO: 130 - protein, 9 aa, Homo sapiens
WGAAVPHSQ

SEQ ID NO: 131 - protein, 9 aa, Homo sapiens
NYSCLDQAR

SEQ ID NO: 132 - protein, 18 aa, Homo sapiens
RKELVETVQCQGSQQPPA

SEQ ID NO: 133 - protein, 16 aa, Homo sapiens
KELVETVQCQGSQQPP

SEQ ID NO: 134 - protein, 9 aa, Homo sapiens
ELVETVQCQ

SEQ ID NO: 135 - protein, 16 aa, Homo sapiens
ASYILIRDTHSLRTTA

SEQ ID NO: 136 - protein, 15 aa, Homo sapiens
SYILIRDTHSLRTTA

SEQ ID NO: 137 - protein, 9 aa, Homo sapiens
ASYILIRDT

(19) United States
(12) Patent Application Publication
(10) Pub. No.: US 2026/0000744 A1
(43) Pub. Date: Jan. 1, 2026

(54) TOLEROGENIC COMPOSITION

(71) Applicants: AHEAD THERAPEUTICS, S.L., BARCELONA (ES); FUNDACIÓ INSTITUT D'INVESTIGACIÓ EN CIÈNCIES DE LA SALUT GERMANS TRIAS I PUJOL (IGTP), BADALONA (ES); FUNDACIÓ HOSPITAL UNIVERSITARI VALL D'HEBRON - INSTITUT DE RECERCA, BARCELONA (ES); UNIVERSITAT DE LLEIDA, LLEIDA (ES)

(72) Inventors: SILVIA RODRIGUEZ VIDAL, BARCELONA (ES); MÍRIAM SALVADÓ MOLERO, VILA-SECA (ES); MONTSERRAT MANCERA ARTEU, SEGUR DE CALAFELL (ES); SILVIA RODRÍGUEZ FERNÁNDEZ, BARCELONA (ES); LIDIA ALMENARA FUENTES, TORRENT (ES); ESTELA ROSELL MASES, LLEIDA (ES); BRUNA BARNEDA ZAHONERO, SANT CUGAT DEL VALLÈS (ES); MARTI DALMASES ARNELLA, SABADELL (ES); BERNABÉ ZEA CHECA, PREMIÀ DE DALT (ES); MARTA VIVES PI, BARCELONA (ES); CARMEN ESPEJO RUIZ, SANT JUST DESVERN (ES); HERENA EIXARCH AHUFINGER, BARCELONA (ES); JUAN VERDAGUER AUTONELL, BARCELONA (ES)

(21) Appl. No.: 19/132,226

(22) PCT Filed: Nov. 22, 2023
(86) PCT No.: PCT/EP2023/082656

§ 371 Date: May 22, 2025

(30) Foreign Application Priority Data
Nov. 23, 2022 (EP) 22383126.4

(51) Int. Cl. A61K 39/00; A61K 9/127; A61K 9/51; A61P 37/06; B82Y 5/00

(52) U.S. Cl. CPC A61K 39/001; A61K 9/127; A61K 9/5123; A61P 37/06; A61K 2039/55555; B82Y 5/00

(57) ABSTRACT

Tolerogenic liposome-based compositions and uses thereof for treating immune disorders. The compositions include two populations of liposomes. The first population of liposomes has a size in the range from 2 to 200 nm, and the second population of liposomes has a size in the range from 500 to 2000 nm. The liposomes of the first and second populations carry one or more antigens. The liposomal membrane of each liposome in the first and second liposome populations include phosphatidylserine in an amount ranging from 20 to 60% by weight with respect to the total composition of the liposome's membrane.

This application claims the benefit of European Patent Application EP22383126.4 filed on 23 Nov. 2022.

### TECHNICAL FIELD

The present disclosure relates to the field of medicine. In particular, the present disclosure provides compositions for inducing tolerance to an antigen, which is useful for the prevention and/or treatment of a variety of immune disorders, such as autoimmune diseases.

### BACKGROUND ART

Abnormal immune responses are responsible for a number of diseases and adverse reactions, including autoimmune diseases, allergies, transplant rejections and drug hypersensitivity.

Autoimmunity, in particular, is the failure of an organism in recognizing its own constituent parts as self, thus leading to an immune response against its own cells and tissues. Prominent examples include type 1 diabetes (T1D), lupus erythematosus, rheumatoid arthritis, multiple sclerosis (MS), Addison's disease, celiac disease, dermatomyositis, Hashimoto's thyroiditis, myasthenia gravis, pernicious anemia, reactive arthritis, Sjogren syndrome, neuromyelitis optica, immune thrombotic thrombocytopenia purpura.

It is calculated that 7 to 10% of the population in developed countries suffers from these diseases, which are often chronic, debilitating, and life-threatening. Autoimmune-associated medical care costs continue to scale up as autoimmune disorders increase world-wide and no effective treatments are made available.

Treatments for autoimmune disease have traditionally been immunosuppressive, anti-inflammatory (steroids), or palliative. Non-immunological therapies, such as hormone replacement in Hashimoto's thyroiditis or Type 1 diabetes mellitus, treat outcomes of the autoaggressive response, thus these are palliative treatments. Dietary manipulation limits the severity of celiac disease. Steroidal or NSAID treatment limits inflammatory symptoms of many diseases. Similar limitations apply to other disorders associated to an abnormal, often excessive, immune response.

Extensive research has been invested in the development of immunomodulating therapies that reduce or avoid an undesired immune response. However, the limited understanding of the intricate details of the different autoimmune diseases substantially slows down progress in this field. Current strategies are generally based on broad-acting immunosuppressive drugs which, in order to maintain immunosuppression, are generally life-long treatments. Additionally, the use of broad-acting immunosuppressants is associated with a risk of severe side effects, such as tumors, infections, nephrotoxicity and metabolic disorders.

Liposomes are lipid vesicles composed of a lipid membrane enclosing an aqueous core. These vesicles are considered to have great potential as drug delivery systems for several reasons, mainly, i) various types of active agents can be delivered; hydrophilic agents can be loaded into the aqueous compartment or hydrophobic agents can be anchored in the membrane, and ii) the therapeutic efficacy may be enhanced and side effects reduces by targeting specific tissues resulting in increased bioavailability of the delivered agent. Liposome-based approaches have been proposed for effectively delivering immunosuppressants in the context of autoimmune diseases with reduced side effects. Additionally, a liposome based approach has been disclosed for inducing tolerance in the absence of immunosuppressants with some success in preventing landmark autoimmune diseases (WO2015107140). The liposomes disclosed in this document are not simple carriers of active agents but they are themselves part of the active agent which is responsible of promoting tolerance directed to the specifically contained antigen(s). The liposomes disclosed in WO2015107140 are relatively big in size (above 500 nm) and mimic apoptotic bodies inducing tolerance of dendritic cells by a mechanism that resembles efferocytosis. However,

In spite of the efforts made until now, there is still ample room for improving the treatment of autoimmune disorders, as well as of other conditions which result from aberrant immune responses.

### SUMMARY OF INVENTION

The inventors have surprisingly found that combining two populations of liposomes carrying an antigen and comprising at least 20% phosphatidylserine in the liposomal membrane, the populations differing in liposome size, achieves a surprising tolerogenic effect which translates into advantageous treatment of immune disorders.

A first aspect of the present disclosure thus relates to a composition comprising two populations of liposomes, wherein:
the first population of liposomes has a size in the range from 2 to 200 nm,
the second population of liposomes has a size in the range from 500 to 2000 nm,
the liposomes of the first and second populations carry one or more antigen, and
the liposomal membrane of each liposome in the first and second liposome populations comprises phosphatidylserine in an amount ranging from 20 to 60% by weight with respect to the total composition of the liposome's membrane.

As shown in the examples below, a composition according to the first aspect induces tolerogenic features (secretion of Interleukine-10 (IL-10) and Transforming Growth Factor Beta (TGF-B)) in human Peripheral Blood Mononuclear Cells (PBMCs). The examples below also show that the composition according to the first aspect, which comprises the two liposomal populations, one having liposome size from 2 to 200 nm and the other having size from 500 to 2000 nm, achieve higher efficacy in ameliorating autoimmune disease than a liposomal composition having the same features except that only the 500-2000 nm population of liposomes is present.

Interestingly, the inventors have surprisingly found that the composition according to the first aspect induces tolerance by interacting with a wide range of antigen presenting cells. Without wanting to be bound by theory, the inventors hypothesize that the liposomal composition herein described may interact with the different players involved in the process of generating peripheral tolerance to achieve a significant effect. Taking into account that different abnormal autoimmune responses may be mediated by different APCs, sometimes more than one APC, the composition of the invention provides an important advantage over other state of the art products in that it has a broader spectrum of action than other products, spanning a broader range of target diseases. Moreover, while generating tolerance through various APCs, the composition of the invention can be seen to act through multiple mechanisms of action, thus achieving a greater effect.

For example, it has been found that the liposomal population of size 2-200 nm is able to induce tolerance in B-cells, while, as disclosed in WO2015107140, the bigger sized population of 500-2000 nm induces tolerance by dendritic cells (DCs). The ability of the smaller size liposome population to interact with B cells and induce tolerogenic features in the same is evidenced in the examples below. For example, FIG. 1 specifically shows that PS-PC-Chol Liposomes under 200 nm interact with B cells and induce IL-10 secretion. Other examples show that the liposomes in the composition of the invention, containing the two populations as described above, interact with and induce tolerogenic features (IL-10 and TGF-B secretion) in several B cells subsets in addition to dendritic cells IL-10 secretion (FIGS. 4, 5 and 6). This effect is surprising in view of WO2015107140, which discloses that liposomal size above 500 nm is essential to mimic apoptotic cells and induce tolerance through dendritic cells via a mechanism that resembles efferocytosis. In view of this prior art document, the effect of the smaller liposome population was not to be expected. Moreover, in view of the teachings of WO2015107140, the expert would not work at liposome size below 500 nm as he would not expect relevant effects for liposomes having this smaller size. Besides other prior art documents disclose use of smaller liposomes for enhancing the immune response (Chen et al, doi:10.4049/jimmunol.1801677). It has also been found that the liposomal composition of the first aspect may induce tolerance through liver sinusoidal endothelial cells (LSEC) and macrophages (FIG. 4).

The tolerogenic effect of the composition as defined in the first aspect of the disclosure (from now on also referred to simply as “liposome composition”) not only results in effective treatment of autoimmune disease, it results in an improved effect when treating autoimmune disease when compared with a liposomal composition comprising only the liposome population sized above 500 nm (FIG. 2).

While being an antigen-specific based therapy, the liposome composition of the first aspect has the advantage of presenting no relevant undesired side effects. As mentioned above, most immunomodulatory approaches for the treatment of autoimmune conditions or transplant rejections involve immunosuppressants, which often lead to high susceptibility to infections and sometimes also promote the development of tumors, nephrotoxicity or metabolic disorders. On top of affording tolerance to the associated antigen, the liposome composition of the first aspect does not induce any toxicity or otherwise undesired side effects.

The liposome composition of the first aspect additionally has several advantages in terms of stability, uniformity, and ease of large-scale production.

First of all, the production of the present liposomes comprising antigens, generally, antigenic peptides, may be achieved using common reagents and equipment in the pharmaceutical industry at a low cost. Moreover, uniformity of the product can be guaranteed, at the same time that scaling-up for large industrial production is affordable and fine-tuning of the dose is facilitated. Besides, since the antigen is protected by the liposome, it is less exposed to degradation.

Another great advantage lies in the fact that the present liposome-based composition is a defined composition, which is devoid of undesired contaminants or by-products. The antigen-containing liposomes do not degenerate into toxic side products, such as necrotic bodies, and do not cause rejections as in the case of autologous or heterologous cell-based therapies.

Another advantage of the herein disclosed liposome composition is that it acts upon two different mechanisms of tolerogenesis. Thus, the efficacy of the treatment is multiplied with virtually no additional side effects. Moreover, the two populations, which provide the dual activity, can be achieved by a single preparation procedure, which is an additional advantage.

Thanks to its tolerogenic effect, the liposome composition as defined in the first aspect affords effective prevention of disorders associated to an abnormal immune response, for example, autoimmune disorders, as well as effective treatment of said disorders, both in a pre-clinical stage (i.e. a stage where the abnormal immune response is already triggered but tissue damage and clinical symptoms are low) and a clinical stage (i.e. a stage where tissue damage is higher and clinical symptoms are evident).

In a second aspect, the present disclosure provides a liposome composition as defined in the first aspect for use as a medicament. This aspect can also be formulated as use of a liposome composition as defined in the first aspect for the preparation of a medicament. Also disclosed is a method of treatment which comprises administering to a subject in need thereof a therapeutically effective amount of a liposome composition as defined in the first aspect.

Without wanting to be bound by theory, it is thought that the therapeutic effect of the liposome composition is achieved through the tolerogenic presentation of the encapsulated antigen by the antigen presenting cells (for example, but not limited to, B-cells, DCs, and macrophages) and subsequent suppression of the abnormal immune response. Thus, in a third aspect, the disclosure provides a liposome composition as defined in the first aspect for use in inducing tolerance. This aspect can also be formulated as use of a liposome composition as defined in the first aspect for the preparation of a medicament for inducing tolerance. Also disclosed is a method for inducing tolerance which comprises administering to a subject in need thereof a therapeutically effective amount of a liposome composition as defined in the first aspect.

In a fourth aspect the disclosure provides a liposome composition as defined to the first aspect for use in the treatment of a disorder associated to an abnormal immune response. This aspect can also be formulated as use of a liposome composition as defined in the first aspect for the preparation of a medicament for treating a disorder associated to an abnormal immune response. Also disclosed is a method for treating a disorder associated to an abnormal immune response which comprises administering to a subject in need thereof a therapeutically effective amount of a composition as defined in the first aspect.

Finally, other aspects of the invention provide a liposome composition as defined above for use in immunomodulation; a liposome composition as defined above for use in suppressing an excessive immune response; and a liposome composition as defined above for use in the treatment of a disorder associated to an abnormal immune response, wherein said liposome composition restores tolerance to the antigen comprised in the liposome composition.

### BRIEF DESCRIPTION OF DRAWINGS

FIG. 1 PS-PC-Chol-Liposomes under 200 nm interact with B cells and induce IL-10 secretion. a % of NBD positive CD19 and CD19CD1d cells from splenocites of NOD mice incubated for 4 hours with PS-PC-Chol-NBD-Ins(h)-liposomes under 200 nm. b %. Of IL-10 expression of B cells from PBMCS after incubation for 24 hours with PS-PC-Chol-Ins(h)-liposomes under 200 nm. Results are presented as mean±SEM.

FIG. 2 PS-PC-Chol-Liposomal composition of the disclosure has higher efficacy than PS-PC-Chol-Liposomes>500 nm. 8 week old mice were immunized with mouse MOG peptide 35-55 (mMOG₃₅₋₅₅), and 5 days post immunization were treated iv with PS-PC-Chol-MOG36-55-liposomes->500 nm or PS-PC-Chol-MOG36-55-liposomes-200&500. Clinical score was assessed for 28 days. Area Under the Curve (AUC) difference of clinical score was calculated to compare the beneficial effect of both liposomal compositions.

FIG. 3 Fluorescent-labeled PS-liposomes are distributed to target organs. a Histogram of relative fluorescent signal (RFU/grams of tissue) in NOD mice 1 and 6h after PS-PC-Chol-AF750-empty-liposomes-200&500 administration. Mice received 100 μl by intravenous route (i.v., n=5). Results are presented as mean±SEM.

FIG. 4 PS-PC-Chol-liposomes-200&500 interact with DCs, B cells, macrophages and LSECs in vivo. Percentages of NBD-fluorescent cells of the parent gate in the spleen and the liver of animals treated with PS-PC-Chol-NBD-Ins(h)-liposomes-200&500 i.v., after 1 h (n=5-6) or 6 h (n=5) of treatment. Cells were determined as follows: B-cells, CD19⁺; conventional DCs (cDCs), CD11c⁺MHC-II⁺CD205⁺CD8a⁺; plasmacytoid DCs (pDCs) CD11c⁺MHC-II⁺B220⁺; macrophages (Mφ), F4/80⁺; Kupffer cells F4/80⁺CD68⁺; Liver sinusoidal endothelial cells (LSEC) CD206⁺F4/80⁻. Data are mean±SEM; differences were found when comparing immune subsets and timepoints within the same subset (*p<0.05, **p<0.01, ***p<0.001, two-way ANOVA with Tukey's multiple comparison test).

FIG. 5 Liposomes of the composition of the invention bind to B cells and induce IL-10 secretion. a Percentage of NBD-fluorescent B-cell subsets within the whole subset gate after PS-PC-Chol-NBD-ins(h)-liposomes-200&500 culture overnight (n=3). Breg-cell subsets were determined as follows: CD19⁺CD1d^{high}CD5⁺; B1a, CD19⁺CD5⁺CD43⁺; B1b, CD19⁺CD5⁻CD43⁺; marginal zone (MZ), CD19⁺CD21^{high}CD23⁻. Data presented as mean±SEM. b Percentage of IL-10-secreting cells from NBD-PSIns(h)-liposome-binding NBD⁺ B-cell subsets (n=3) and from non-liposome-binding NBD⁻ B-cell subsets (n=3). Data presented as mean±SEM; no differences were found.

FIG. 6 Liposomes of the composition of the invention induce the tolerogenic cytokines IL-10 and TGF-B expression in B and IL-10 expression in DCs cells from PBMCs. a % Of IL-10 expression of B cells NBD+ or NBD− from PBMCS after incubation for 24 hours with PS-PC-Chol-NBD-liposomes-200&500 loaded with AChR peptide or mutated citrullinated vimentin peptide or insulin. b % Of TGF-b expression of B cells NBD+ or NBD− from PBMCS after incubation for 24 hours with PS-PC-Chol-NBD-liposomes-200&500 loaded with AChR peptide or mutated citrullinated vimentin peptide or insulin. c % Of IL-10 expression of Dendritic Cells NBD+ or NBD− from PBMCS after incubation for 24 hours with PS-PC-Chol-NBD-liposomes-200&500 loaded with AChR peptide or mutated citrullinated vimentin peptide or insulin. d % Of TGF-b expression of Dendritic Cells NBD+ or NBD− from PBMCS after incubation for 24 hours with PS-PC-Chol-NBD-liposomes-200&500 loaded with AChR peptide or mutated citrullinated vimentin peptide or insulin. e % of pHrodo green positive cells of the parent gate of different immune cell types from PBMCs after incubation for 24 hours with PS-PC-Chol-MCV-200&500 stained with pHrodo™ green Dye. f IL-10 and TGF-b expression of B cells pHrodo+ and pHrodo− expressed by mean fluorescence intensity (MFI) Results are presented as mean±SEM. (*p<0.05, **p<0.01, ***p<0.001, two-way ANOVA with Tukey's multiple comparison test)

### DETAILED DESCRIPTION OF THE INVENTION

Unless defined otherwise, all technical and scientific terms used herein have the same meaning as is commonly understood by one with skill in the art to which this invention belongs at the time of filling. However, in the event of any latent ambiguity, definitions provided herein take precedent over any dictionary or extrinsic definition. Further, unless otherwise required by context, singular terms shall include pluralities and plural terms shall include the singular.

As used herein, the indefinite articles “a” and “an” are synonymous with “at least one” or “one or more.” Unless indicated otherwise, definite articles used herein, such as “the” also include the plural of the noun.

### Liposomes

As mentioned above, the first aspect of the disclosure relates to a composition comprising two populations of liposomes, wherein the first population of liposomes has a size in the range from 2 to 200 nm and the second population of liposomes has a size in the range from 500 to 2000 nm. In a particular embodiment, the composition of the first aspect consists essentially of two populations of liposomes, wherein the first population of liposomes has a size in the range from 2 to 200 nm and the second population of liposomes has a size in the range from 500 to 2000 nm. In another particular embodiment, the composition of the first aspect consists of two populations of liposomes, wherein the first population of liposomes has a size in the range from 2 to 200 nm and the second population of liposomes has a size in the range from 500 to 2000 nm. The liposomes of the first and second populations may carry one or more than one type of antigen. The liposomal membrane of each liposome in the first and second liposome populations may comprise phosphatidylserine in an amount ranging from 20 to 60% by weight with respect to the total composition of the liposome's membrane.

The term “liposome” is to be understood as a self-assembling structure comprising one or more membranes comprised by amphipathic bilayers, each of which comprises two monolayers containing amphipathic molecules oppositely oriented. Amphipathic molecules, such as amphipathic lipids, comprise a polar (hydrophilic) headgroup region covalently linked to one or two non-polar (hydrophobic) chains. Energetically unfavorable contacts between the hydrophobic chains and the surrounding aqueous medium induce the amphipathic molecules to arrange themselves such that their polar headgroups are oriented towards the bilayer's surface, while the hydrophobic chains reorient towards the interior of the bilayer. An energetically stable structure is thus formed in which the hydrophobic chains are effectively shielded from coming into contact with the aqueous environment.

Liposomes can have a single bilayer membrane (small unilamellar vesicles “SUVs” and large unilamellar vesicles “LUVs”), or multiple bilayer membrane (multilamellar large vesicles “MLVs”). Liposomes may also be prepared as multivesicular vesicles “MVVs”, which are liposomes enclosing, or encapsulating, multiple non-concentric aqueous chambers. In contrast, MLVs have multiple concentric “onion-skin”-like membranes, each of which encapsulates an aqueous compartment. Given this encapsulation of aqueous volume within a protective barrier of lipid molecules, liposomes are able to sequester encapsulated molecules, e.g., peptides, away from the degrading effects of factors, e.g., peptidase enzymes, present in the external environment. The sequestered molecules may be suspended or dissolved in the aqueous compartment(s) or associated to the liposomal membrane(s). In general, polar, water-soluble molecules will be mainly dissolved in the aqueous compartment(s), while less polar molecules may be associated to the lipidic membrane.

The liposome composition of the first aspect comprises two populations of liposomes, wherein the first population of liposomes has a size in the range from 2 to 200 nm and the second population of liposomes has a size in the range from 500 to 2000 nm. The size of the liposomes usually refers to the mean diameter and may be determined by Nanoparticle Tracking Analysis (NTA) using a Nanosight NS300 from Malvern Panalytical (Whitepaper on Nanoscale Material Characterization: a Review of the use of Nanoparticle Tracking Analysis (NTA), 2015 Malvern Instruments Limited). This method is known to determine the “hydrodynamic diameter”. Thus, in particular embodiments of the disclosure, the size of the liposomes refers to the hydrodynamic diameter. The size of the liposomes in the second population is not usually over 2000 but, in some embodiments, the size range of this second population can be larger, for example, from 500 to around 3000, 4000 or 5000 nm.

In one embodiment, the liposomes of the first population have a size below 200 nm, or below 180 nm, or below 160 nm, or below 150 nm, or below 125 nm, or below 100 nm, or below 75 nm, or below 50 nm. In another embodiment, the liposomes of the first population have a size above 2 nm, or above 10 nm, or above 15nm, or above 25 nm, or above 50 nm, or above 75 nm. In a particular embodiment the liposomes of the first population have a size in the range from 2 to 200 nm, more particularly from 10 to 200 nm.

In one embodiment, the liposomes of the second population have a size above 500 nm, or above 525 nm, or above 550 nm, or above 575 nm, or above 600 nm, or above 625 nm, or above 650 nm, or above 675 nm, or above 700 nm. In another embodiment, the liposomes of the second population have a size below 5000 nm, or below 4000 nm, or below 3000 nm, or below 2000 nm, or below 1800 nm, or below 1500 nm, or below 1250 nm, or below 1100 nm, or below 1000 nm, or below 950 nm, or below 900 nm, or below 850 nm, or below 800 nm.

In one embodiment, 15-75% of the liposomes in the composition correspond to the first population, i.e., 15-75% of the liposomes in the composition have a size in the range from 2 to 200 nm. These percentages refer to the number of liposomes having the specified size per 100 liposomes in the composition. In a particular embodiment, 15-55% of the liposomes in the composition correspond to the first population. In a more particular embodiment, 20-50% of the liposomes in the composition correspond to the first population. In one embodiment, 2-40% of the liposomes in the composition correspond to the second population, i.e. 2-40% of the liposomes in the composition have a size in the range from 500 to 2000 nm. In a particular embodiment, 2-25% of the liposomes in the composition correspond to the second population. In another more particular embodiment, 2-15% of the liposomes in the composition correspond to the second population.

As mentioned above, the liposomal membrane is formed by amphipathic molecules. Non-limiting amphipathic molecules that may be contained in the liposomal membrane of the liposome composition of the present disclosure include, phospholipids; glycerophospholipids; phosphatidyilserines (PS), such as 1,2-dioleoyl-phosphatidylserine, 1,2-dioleoyl-sn-glycero3-phospho-L-serine (DOPS), 1,2-dipalmitoyl-phosphatidylserine, 1,2-dimyristoyl-phosphatidylserine, 1,2-distearoyl-phosphatidylserine, I-oleoyl-2-palmitoyl-phosphatidylserine, I-oleoyl-2-stearoyl-phosphatidylserine, 1-palmitoyl-2-oleoyl-phosphatidylserine, and I-stearoyl-2-oleoyl-phosphatidylserine; phosphatidylcholines (PC), such as 1,2-Dimyristoyl-sn-glycero-3-phosphocholine (DMPC), dipalmitoyl phosphatidylcholine (DPPC), dioleoylphosphatidylcholine, lecithin; lysolecithin; phosphatidylethanolamines, such as dioleylphosphatidyl ethanolamine (DOPE); poly(ethylene glycol)5000-phosphatidylethanolamine; dioleyloxypropyltriethylammonium (DOTMA); cholesterol; cholesterol ester; diacylglycerol; diacylglycerol succinate; diphosphatidyl glycerol (DPPG); hexanedecanol; fatty alcohols such as polyethylene glycol (PEG); poly(ethylene glycol)400-monostearate; polyoxyethylene-9-lauryl ether; a surface active fatty acid, such as palmitic acid or oleic acid; fatty acids; fatty acid monoglycerides; fatty acid diglycerides;a sorbitan fatty acid ester such as sorbitan trioleate; phosphatidylinositol; sphingomyelin; cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate and combinations thereof.

In one embodiment, the liposome membrane may include, without limitation, phospholipids such as phosphatidylcholine (PC), phosphatidyilserine (PS), phosphatidylethanolamine (PE), phosphatidylglycerol (PG), phosphatidylinositol (PI) and phosphatidic acid (PA). The liposomal membrane may also include other lipids, such as cholesterol (CHOL). The liposome membrane may contain additional molecules not lipidic in nature, such as proteins, carbohydrates, antibodies or polyethylene glycol (PEG) chains. The liposomes may include particular moieties which are designed to target the liposome to a specific site or target cell, or protect the liposome against a hostile environment (e.g. the gastrointestinal tract). The composition of the liposome is relevant for tolerogenic delivery of the antigen. Thus, as mentioned above, the liposome membrane preferably comprises PS in an amount ranging from 20 to 60% by weight with respect to the total composition of the liposome's membrane. PS contained in the liposomal membrane constitutes an ‘eat me’ signal that connects PS-recognition by antigen presenting cells with the consequences in tolerance induction. It is noteworthy that the liposome of the invention does not require of further receptors or ligands to be effectively engulfed by antigen-presenting cells and achieve a tolerogenic delivery of the antigen. However, other receptors and/or ligands may be assembled into the liposome in order to improve uptake and/or tolerogenic processing.

The term “percentage (%) by weight” refers to the percentage of each component of the liposome's membrane in relation to the total weight of the liposome's membrane.

By “liposome's membrane” or “liposomal membrane” it is referred to the totality of membrane bilayers contained in the liposomes.

In one embodiment the liposomal membrane comprises PS in an amount comprised from 30 to 50% by weight with respect to the total liposomal membrane. In further embodiments, the liposomal membrane comprises PS in an amount comprised from 30 to 45%, or from 35 to 45%, by weight with respect to the total liposomal membrane, for example 36%, 37%, 38%, 39%, 40%, 41%, 42%, 43%, or 44%.

The liposome according to the present disclosure may comprise, in addition to PS, variable concentrations of other lipids. In some embodiments, the liposomal membrane also comprises phospholipids with a neutral net charge. In some embodiments, the liposomal membrane also comprises phosphatidylethanolamine, 1,2-dilinoleoyl-sn-glycero-3-phosphoethanolamine, 1,2-dilinoleoyl-sn-glycero-3-phosphocholine, 1-stearoyl-2-linoleoyl-sn-glycero-3-phosphocholine, 1-stearoyl-2-oleoyl-sn-glycero-3-phosphocholine, and/or sphingomyelin. In some embodiments, the liposomal membrane also comprises PC, for example 1,2-Dimyristoyl-sn-glycero-3-phosphocholine (DMPC). In some embodiments, the liposomal membrane also comprises sterol lipids or steroids. In particular embodiments the liposomal membrane also comprises PC and CHOL.

In one embodiment the liposomal membrane comprises PC in an amount comprised from 30 to 50% by weight with respect to the total liposomal membrane. In another embodiment the liposome's membrane comprises PC in an amount comprised from 30 to 45% by weight with respect to the total liposomal membrane. In further embodiments, membrane PC is comprised from 32 to 42% by weight with respect to the total liposomal membrane, for example 33%, 34%, 35%, 36%, 37%, 38%, 39%, 40%, or 41%.

The amount of CHOL may be comprised from 10 to 40% by weight with respect to the total liposomal membrane. In one embodiment, the liposome's membrane comprises CHOL in an amount comprised from 12 to 40% by weight with respect to the total liposomal membrane. In further embodiments, membrane CHOL is comprised from 15 to 37%, or from 20 to 35%, or from 20 to 30% by weight with respect to the total liposomal membrane, for example 21%, 22%, 23%, 24%, 25%, 26%, 27%, 28%, or 29%.

The proportion of the different lipids contained in the liposome membrane must be equilibrated in order to obtain a liposome with appropriate physical and chemical properties in terms of stability, permeability and morphology. In some embodiments, the liposomal membrane comprises PS, PC and CHOL. In some embodiments, the liposomal membrane consists essentially of PS, PC and CHOL. In some embodiments, the liposomal membrane consists of PS, PC and CHOL. In some embodiments, the molar ratio PS:PC:CHOL is 1:(0.2-4):(0.2-5). In particular embodiments, the molar ratio PS:PC:CHOL is 1:(0.6-1.8):(0.7-2.5). The term “ratio” is understood in the usual sense as the magnitude of quantities relative to each other. Specifically, the ratio of two quantities indicates how many times the first quantity (X) is contained in the second quantity (Y), and is expressed as X:Y. As in the embodiment above, the quantities may be expressed as a range (X-X′:Y-Y′). The term “molar ratio” is used when the referred magnitude is the molarity. Alternatively, the ratio may be expressed as “weight ratio” when the referred magnitude is weight. Here, ranges of molar ratios are given for the three particular lipids (PS, PC and CHOL). In a more particular embodiment, the membrane comprises a molar ratio PS:PC:CHOL which is 1:(0.7-1.5):(0.9-2). In another more particular embodiment, the membrane comprises a molar ratio PS:PC:CHOL which is 1:(0.8-1.4):(1.1-1.9). In another more particular embodiment, the membrane comprises a molar ratio PS:PC:CHOL which is 1:(0.9-1.3):(1.2-1.7).

### Antigens

As mentioned above, the liposomes comprised in the composition of the first aspect comprise an antigen. The term “antigen” refers to any substance that generates B-cell (humoral/antibody) and/or T-cell (cellular) adaptive immune responses upon exposure to a host organism. The antigens may be dissolved or suspended in the aqueous compartment(s) inside the liposome or associated to the liposomal membrane. An antigen is a molecule that can bind to the components of the immune system, including antibodies, T cells, and B cells. A particular antigen may contain one or more epitopes or antigen determinants. In the sense of the present disclosure, antigens are the immunogenic antigens. The skilled person is able to determine antigenic substances by methods that are well-known in the field.

The liposome composition of the first aspect may contain one type of antigen or more than one type of antigen. In certain embodiments, the liposome encapsulates more than one type of antigen associated with the same immunological disorder. For example, the liposome may encapsulate two, three, four or five different antigens, all of which are preferably associated with the same immunological disorder.

Antigens may have different chemical nature. Most antigens are of peptidic nature, however, polysaccharides, lipids or nucleic acids can also be antigenic molecules. Moreover, some antigens are complexes, such as complexes formed by polypeptides and other molecules selected from nucleic acids, lipids and polysaccharides. For example, the disclosure contemplates that the liposomes contain polypeptides comprising one or more antigenic sequence. When the liposomes contain more than one different antigen, the disclosure contemplates that the different antigens are independent antigenic peptide sequences. It is also contemplated that the different antigens form part of one discrete sequence which comprises more than one different antigenic sequence. The antigens (antigenic sequences) may be located consecutively within the polypeptide or, alternatively, they can be separated by non-antigenic, linking sequences. The present disclosure is not limited to any particular antigen or group of antigens. The liposome composition of the present disclosure can be prepared by including any antigen and elicits antigen-specific tolerogenicity against any antigen contained therein.

In one embodiment the antigen is selected from the group consisting of self-antigens, drugs, including therapeutic proteins, allergens and alloantigens.

In one embodiment, the antigen is a peptide. In a particular embodiment, the antigen is a peptide having a size in the rage from 3 to 5000 amino acids, particularly from 5 to 2000 amino acids, or from 6 to 1000 amino acids, or from 7 to 500 amino acids, or from 8 to 250 amino acids, or from 5 to 200 amino acids, or from 8 to 100 amino acids, or from 10 to 50 amino acids. In a particular embodiment, the antigen is a peptide having a size in the rage from 5 to 100 amino acids.

In one embodiment of the first aspect, the antigen is a self-antigen. The term “self-antigen” generally refers to a normal substance, often a or complex of proteins, that is recognized by the immune system of patients suffering from a specific autoimmune disease. These antigens should, under normal conditions, not be the target of the immune system, but, due to mainly genetic and environmental factors, the normal immunological tolerance for such an antigen has been lost in these patients.

In one embodiment of the first aspect, the antigen is a self-antigen associated to type 1 diabetes (T1D), lupus erythematosus, rheumatoid arthritis, juvenile rheumatoid arthritis, osteoarthritis, reactive arthritis, psoriatic arthritis, multiple sclerosis, neuromyelitis optica, Addison's disease, celiac disease, dermatomyositis, Hashimoto's thyroiditis, myasthenia gravis, pernicious anemia, autoimmune hemolitic anemia, autoimmune neutrophenia, Graves' disease, psoriasis, Sjogren syndrome, including keratoconjunctivitis sicca secondary to Sjogren's Syndrome, autoimmune thyroiditis, dermatitis (including atopic dermatitis and eczematous dermatitis), alopecia areata, Crohn's disease, aphthous ulcer, iritis, conjunctivitis, keratoconjunctivitis, ulcerative colitis, autoimmune uveitis, allergic encephalomyelitis, acute necrotizing hemorrhagic encephalopathy, idiopathic bilateral progressive sensorineural hearing loss, aplastic anemia, pure red cellanemia, idiopathic thrombocytopenia, immune-mediated thrombotic thrombocytopenia purpura, polychondritis, Wegener's granulomatosis, chronic active pepatitis, Stevens Johnson syndrome, idiopathic sprue, lichen planus, sarcoidosis, primary biliary cirrhosis, uveitis posterior, and interstitial lung fibrosis.

In another embodiment of the first aspect, the antigen is a self-antigen. Self-antigens (also called autoantigens) are known to the skilled person and can be found in the literature. For example, self-antigens associated to a myriad of autoimmune disease are disclosed in the AAgAtlas 1.0 database (doi: 10.1093/nar/gkw946. Epub 2016 Oct. 19; http://biokb.ncpsb.org/aagatlas/).

In a particular embodiment the self-antigen is selected from the group consisting of insulin, proinsulin, protein tyrosine phosphatase (IA2, also known as islet cell antigen 512), glutamate decarboxylase (GAD), chromogranin and islet-glucose-6-phosphatase catalytic subunit-related protein (IGRP), peripherin (Roep B O, Peakman M. Cold Spring Harb Perspect Med, 2012, vol. 2(4):a007781. oi: 10.1101/cshperspect.a007781.), myelin, myelin-oligodendrocyte glycoprotein (MOG), myelin basic protein (MBP), myelin proteolipid protein (PLP), GDP-I-fucose synthase, acetylcholine receptor (AChR), Muscle-specific tyrosine kinase (MuSK), Agrin, lipoprotein related protein 4 (LRP4), cortactin, transglutaminase, deamidated gliadin, thyroglobulin (Lernmark A. J. Clin Invest, 2001, vol. 108, p. 1091-1096), collagen (e.g., collagen type 11), human cartilage gp 39, chromogranin A, gp130-RAPS, vimentin, citrullinated vimentin, ADAMST13, aquaporin-4, proteolipid protein, fibrillarin, nuclear proteins, nucleolar proteins (e.g., small nucleolar protein), histidyl-tRNA synthetase (HisRS), histidine-tRNA synthetase (HARS1), jo-1, thyroid stimulating factor receptor, histones, glycoprotein gp 70, ribosomal proteins, pyruvate dehydrogenase dehydrolipoamide acetyltransferase, hair follicle antigens, human tropomyosin isoform 5, mitochondrial proteins, pancreatic β-cell proteins, gluten, and antigenic fragments or derivatives of any of the above. Examples of antigenic fragments are, for instance, those disclosed in table 3.

In other embodiments the self-antigen is associated to an autoimmune disorder selected from those disclosed in table 1. In other embodiments the self-antigen is a polypeptide selected from those disclosed in table 1 and antigenic fragments of said polypeptides. In one particular embodiment the antigen is associated with T1D. In a more particular embodiment the antigen is selected from those disclosed in table 2 and combinations or immunogenic fragments thereof.

TABLE 1

| Autoantigen | Autoimmune disease or disorder |
|---|---|
| ribonucleoproteins | Systemic lupus erythematosus, neonatal heart block, |
| | primary Sjögren's syndrome |
| ribonucleoproteins | Primary Sjögren's syndrome |
| centromere | CREST syndrome |
| Ri | Opsoclonus |
| double-stranded DNA | SLE |
| histidine-tRNA ligase | Inflammatory myopathy |
| snRNP core proteins | SLE |
| Type I topoisomerase | Systemic sclerosis (anti-Scl-70 antibodies) |
| histones | SLE and Drug-induced LE |
| nucleoporin 62 Sp100 nuclear | Primary biliary cirrhosis |
| antigen | |
| nucleoporin 210 kDa | |
| | Coeliac disease |
| | Dermatitis herpetiformis |
| ganglioside GQ1B | Miller-Fisher Syndrome |
| ganglioside GD3 | Acute motor axonal neuropathy (AMAN) |
| ganglioside GM1 | Multifocal motor neuropathy with conduction block (MMN) |
| actin | Coeliac disease anti-actin antibodies have been correlated with the |
| | level of intestinal damage |
| | Autoimmune hepatitis |
| thrombin | Systemic lupus erythematosus |
| phospholipid proteins | Antiphospholipid syndrome |
| in neutrophil cytoplasm | |
| | Wegener's granulomatosis |
| neutrophil perinuclear | Microscopic polyangiitis, Churg-Strauss syndrome, systemic |
| | vasculitides (non- specific) |
| smooth muscle | Chronic autoimmune hepatitis |
| mitochondria | Primary biliary cirrhosis |
| signal recognition particle | Polymyositis |
| exosome complex | Scleromyositis |
| nicotinic acetylcholine receptor | Myasthenia gravis |
| muscle-specific kinase (MUSK) | Myasthenia gravis |
| voltage-gated calcium | Lambert-Eaton myasthenic syndrome |
| channel(P/Q-type) | |
| thyroid peroxidase(microsomal) | Hashimoto's thyroiditis |
| TSH receptor | Graves' disease |
| Hu | Paraneoplastic cerebellar syndrome |
| Yo (cerebellar Purkinje Cells) | Paraneoplastic cerebellar syndrome |
| amphiphysin | Stiff person syndrome, paraneoplastic cerebellar syndrome |
| voltage-gated potassium channel | Limbic encephalitis, Isaac's Syndrome(autoimmune neuromyotonia) |
| (VGKC) | |
| basal ganglia neurons | Sydenham's chorea, paediatric autoimmune neuropsychiatric |
| | disease associated with Streptococcus (PANDAS) |
| N-methyl-D- aspartate receptor | Encephalitis |
| (NMDA) | |
| glutamic acid decarboxylase | Diabetes mellitus type 1, stiff person syndrome |
| (GAD) | |
| aquaporin-4 | Neuromyelitis optica (Devic's syndrome) |

TABLE 2
T1D self-antigens

| Autoantigen | Tissue distribution |
|---|---|
| Preproinsulin/Proinsulin/Insulin | β-cells, thymus |
| Amylin | β-cells |
| IGRP | β-cells |
| ZnT8 | β-cells |
| GAD65 | β-cells, adrenal gland, CNS, neurons, |
| | testis, ovary |
| GAD67 | β-cells (weakly), neurons |
| IA-2 | β-cells |
| IA-2β | β-cells |
| ICA-69 | Islet cells, heart, brain |
| Imogen 38 | Islet cells |
| PDX1 | β-cells |
| Carboxipeptidase H/E | β-cells, neuroendocrine cells, adrenals |
| HSP60 | Ubiquitous (mitochondria) |
| Sulfatide | Ubiquitous |
| Ganglioside GM2-1 | Islet cells |
| ICA69 | β-cells and neurons |
| HSP70 | Ubiquitous |
| Peripherin | Neuroendocrine cells |
| REG1A | Islet cells |
| Cromogranina A | Neuroendrocrine cells |
| IAPP | β-cells |

IGRP: glucose-6-phosphatase catalytic subunit-related protein; ZnT8: zinc transporter 8, GAD: glutamic acid decarboxylase; IA-2: insulinoma-antigen 2; ICA-69: islet cell antigen-69; PDX1: pancreatic duodenal homeobox protein 1; HSP60: heat shock protein 60.

TABLE 3
Antigenic fragments

| | SEQ ID | Sequence |
|---|---|---|
| Derived | SEQ ID NO: 1 | MASLSRPSLPSCLCSFLLLLLLQVSSSYAGQFRVIGPRHPIRALVGDEVELPCRIS |
| from MOG | | PGKNATGMEVGWYRPPFSRWHLYRNGKDQDGDQAPEYRGRTELLKDAIGEG |
| | | KVTLRIRNVRFSDEGGFTCFFRDHSYQEEAAMELKVEDPFYWVSPGVLVLLAVL |
| | | PVLLLQITVGLIFLCLQYRLRGKLRAEIENLHRTFDPHFLRVPCWKITLFVIVPVLGP |
| | | LVALIICYNWLHRRLAGQFLEELRNPF |
| | SEQ ID NO: 2 | MEVGWYRPPFSRVVHLYRNGK |
| | SEQ ID NO: 3 | MEVGWYRSPFSRVVHLYRNGK |
| | SEQ ID NO: 4 | EVGWYRSPFSRVVHLYRNGK |
| | SEQ ID NO: 5 | GQFRVIGPRHPIRALVGDEV |
| | SEQ ID NO: 6 | DEGGYTCFFRDHSYQ |
| Derived | SEQ ID NO: 7 | MGLLECCARCLVGAPFASLVATGLCFFGVALFCGCGHEALTGTEKLIETYFSKNY |
| from PLP | | QDYEYLINVIHAFQYVIYGTASFFFLYGALLLAEGFYTTGAVRQIFGDYKTTICGKG |
| | | LSATVTGGQKGRGSRGQHQAHSLERVCHCLGKWLGHPDKFVGITYALTVVWLL |
| | | VFACSAVPVYIYFNTWTTCQSIAFPSKTSASIGSLCADARMYGVLPWNAFPGKVC |
| | | GSNLLSICKTAEFQMTFHLFIAAFVGAAATLVSLLTFMIAATYNFAVLKLMGRGTKF |
| | SEQ ID NO: 8 | HCLGKWLGHPDKF |
| | SEQ ID NO: 9 | NTWTTSQSIAFPSK |
| | SEQ ID NO: 10 | HCLGKWLGHPDKFVGITYALTWWWLLVFACSAVPVYIYFNTWTTCQSIAFPS |
| | SEQ ID NO: 11 | HCLGKWLGHPDKFVGI |
| Derived | SEQ ID NO: 12 | MGNHAGKRELNAEKASTNSETNRGESEKKRNLGELSRTTSEDNEVFGEADANQ |
| from MBP | | NNGTSSQDTAVTDSKRTADPKNAWQDAHPADPGSRPHLIRLFSRDAPGREDNT |
| | | FKDRPSESDELQTIQEDSAATSESLDVMASQKRPSQRHGSKYLATASTMDHARH |
| | | GFLPRHRDTGILDSIGRFFGGDRGAPKRGSGKDSHHPARTAHYGSLPQKSHGRT |
| | | QDENPWHFFKNIVTPRTPPPSQGKGRGLSLSRFSWGAEGQRPGFGYGGRASD |
| | | YKSAHKGFKGVDAQGTLSKIFKLGGRDSRSGSPMARR |
| | SEQ ID NO: 13 | KYLATASTMDHARHGFLPRH |
| | SEQ ID NO: 14 | AQGTLSKIFKLGGRDSRSGSPMARR |
| | SEQ ID NO: 15 | VHFFKNIVTPRTPPPSQGKGR |
| | SEQ ID NO: 16 | LSRFSWGAEGQRPGFGYGG |
| | SEQ ID NO: 17 | ENPVVHFFKNIVTPRTP |
| IGRP | SEQ ID NO: 18 | LHRSGVLIIHHLQEDYRTY |
| | SEQ ID NO: 19 | KYNKANVFL |
| Derived | SEQ ID NO: 20 | MALWMRLLPLLALLALWGPDPAAAFVNQHLCGSHLVEALYLVCGERGFFYTPKT |
| from pre-pro | | RREAEDLQVGQVELGGGPGAGSLQPLALEGSLQKRGIVEQCCTSICSLYQLENY |
| insulin or | | CN |
| insulin | SEQ ID NO: 21 | SHLVEALYLVCGERGFFYTPKTRREAEDLQVGQVELGGGPGAGSLQPLALEGSL |
| | | QSRG |
| | SEQ ID NO: 22 | SHLVEALYLVCGERG |
| | SEQ ID NO: 23 | GIVEQCCTSICSLYQLENYCN |
| | SEQ ID NO: 24 | FVNQHLCGSHLVEALYLVCGERGFFYTPKT |
| | SEQ ID NO: 25 | EAEDLQVGQVELGGGPGAGSLQPLALEGSLQ |
| | SEQ ID NO: 26 | GAGSLQPLALEGSLQKRG |
| | SEQ ID NO: 27 | SLQPLALEGSLQSRG |
| Deamidated | SEQ ID NO: 28 | PQPEQPF |
| gliadin | SEQ ID NO: 29 | PQPELPY |
| peptides | SEQ ID NO: 30 | EQEQP |
| | SEQ ID NO: 31 | EGSFQPSQE |
| | SEQ ID NO: 32 | LQLQPFPQPELPYPQPQLPYPQPELPYPQPQPF |
| Vimentin | SEQ ID NO: 33 | MSTRSVSSSSYRRMFGGPGTASRPSSSRSYVTTSTRTYSLGSALRPSTSRSLYA |
| derived | | SSPGGVYATRSSAVRLRSSVPGVRLLQDSVDFSLADAINTEFKNTRTNEKVELQE |
| peptides | | LNDRFANYIDKVRFLEQQNKILLAELEQLKGQGKSRLGDLYEEEMRELRRQVDQL |
| | | TNDKARVEVERDNLAEDIMRLREKLQEEMLQREEAENTLQSFRQDVDNASLARL |
| | | DLERKVESLQEEIAFLKKLHEEEIQELQAQIQEQHVQIDVDVSKPDLTAALRDVRQ |
| | | QYESVAAKNLQEAEEWYKSKFADLSEAANRNNDALRQAKQESTEYRRQVQSLT |
| | | CEVDALKGTNESLERQMREMEENFAVEAANYQDTIGRLQDEIQNMKEEMARHL |
| | | REYQDLLNVKMALDIEIATYRKLLEGEESRISLPLPNFSSLNLRETNLDSLPLVDTH |
| | | SKRTLLIKTVETRDGQVINETSQHHDDLE |
| | SEQ ID NO: 34 | RPSTSRSLYASSPGGVYATRSSAVRLRSSVPGVR |
| | SEQ ID NO: 35 | PGGVYATRSSAVRLRSSVPGVR |
| | SEQ ID NO: 36 | MSTRSVSSSSYRRMFGGPGTASRPSSSRSYVTTSTRTYSLGSALRPSTSRSLYA |
| | | SSPGGVYATXSSAVXLXSSVPGVXLLQDSVDFSLADAINTEFKNTRTNEKVELQE |
| | | LNDRFANYIDKVRFLEQQNKILLAELEQLKGQGKSRLGDLYEEEMRELRRQVDQL |
| | | TNDKARVEVERDNLAEDIMRLREKLQEEMLQREEAENTLQSFRQDVDNASLARL |
| | | DLERKVESLQEEIAFLKKLHEEEIQELQAQIQEQHVQIDVDVSKPDLTAALRDVRQ |
| | | QYESVAAKNLQEAEEWYKSKFADLSEAANRNNDALRQAKQESTEYRRQVQSLT |
| | | CEVDALKGTNESLERQMREMEENFAVEAANYQDTIGRLQDEIQNMKEEMARHL |
| | | REYQDLLNVKMALDIEIATYRKLLEGEESRISLPLPNFSSLNLRETNLDSLPLVDTH |
| | | SKRTLLIKTVETRDGQVINETSQHHDDLE |
| | SEQ ID NO: 37 | RPSTSXSLYASSPGGVYATXSSAVXLRSSVPGVR |
| | SEQ ID NO: 38 | RPSTSXSLYASSPGGVYATXSSAVRLRSSVPGVR |
| | SEQ ID NO: 39 | RPSTSXSLYASSPGGVYATRSSAVXLRSSVPGVR |
| | SEQ ID NO: 40 | RPSTSRSLYASSPGGVYATXSSAVXLRSSVPGVR |
| | SEQ ID NO: 41 | RPSTSXSLYASSPGGVYATRSSAVRLRSSVPGVR |
| | SEQ ID NO: 42 | RPSTSRSLYASSPGGVYATXSSAVRLRSSVPGVR |
| | SEQ ID NO: 43 | RPSTSRSLYASSPGGVYATRSSAVXLRSSVPGVR |
| | SEQ ID NO: 44 | PGGVYATXSSAVXLRSSVPGVX |
| | SEQ ID NO: 45 | PGGVYATXSSAVRLRSSVPGVX |
| | SEQ ID NO: 46 | PGGVYATXSSAVXLRSSVPGVR |
| | SEQ ID NO: 47 | PGGVYATRSSAVXLRSSVPGVX |
| | SEQ ID NO: 48 | PGGVYATXSSAVRLRSSVPGVR |
| | SEQ ID NO: 49 | PGGVYATRSSAVXLRSSVPGVR |
| | SEQ ID NO: 50 | PGGVYATRSSAVRLRSSVPGVX |
| jo-1 derived | SEQ ID NO: 51 | MAERAALEELVKLQGERVRGLKQQKASAELIEEEVAKLLKLKAQLGPDESKQKFV |
| peptides | | LKTPKGTRDYSPRQMAVREKVFDVIIRCFKRHGAEVIDTPVFELKETLMGKYGED |
| | | SKLIYDLKDQGGELLSLRYDLTVPFARYLAMNKLTNIKRYHIAKVYRRDNPAMTRG |
| | | RYREFYQCDFDIAGNFDPMIPDAECLKIMCEILSSLQIGDFLVKVNDRRILDGMFAI |
| | | CGVSDSKFRTICSSVDKLDKVSWEEVKNEMVGEKGLAPEVADRIGDYVQQHGG |
| | | VSLVEQLLQDPKLSQNKQALEGLGDLKLLFEYLTLFGIDDKISFDLSLARGLDYYT |
| | | GVIYEAVLLQTPAQAGEEPLGVGSVAAGGRYDGLVGMFDPKGRKVPCVGLSIGV |
| | | ERIFSIVEQRLEALEEKIRTTETQVLVASAQKKLLEERLKLVSELWDAGIKAELLYK |
| | | KNPKLLNQLQYCEEAGIPLVAIIGEQELKDGVIKLRSVTSREEVDVRREDLVEEIKR |
| | | RTGQPLCIC |
| | SEQ ID NO: 52 | MAERAALEELVKLQGERVRGLKQQKASAELIEEEVAKLLKLKAQLGPDESKQKFV |
| | | LKTPKGTRDYSPRQMAVREKVFDVIIRCFKRHGAEVIDTPVFELKETLMGKYGED |
| | | SKLIYDLKDQGGELLSLRYDLTVPFARYLAMNKLTNIKRYH |
| ACHR | SEQ ID NO: 53 | MEPWPLLLLFSLCSAGLVLGSEHETRLVAKLFKDYSSWVRPVEDHRQVVEVTVG |
| derived | | LQLIQLINVDEVNQIVTTNVRLKQQWVDYNLKWNPDDYGGVKKIHIPSEKIWRPDL |
| peptides | | VLYNNADGDFAIVKFTKVLLQYTGHITWTPPAIFKSYCEIIVTHFPFDEQNCSMKLG |
| | | TWTYDGSVVAINPESDQPDLSNFMESGEWVIKESRGWKHSVTYSCCPDTPYLDI |
| | | TYHFVMQRLPLYFIVNVIIPCLLFSFLTGLVFYLPTDSGEKMTLSISVLLSLTVFLL |
| | | VIVELIPSTSSAVPLIGKYMLFTMVFVIASIIITVIVINTHHRSPSTHVMPNWVRKV |
| | | FIDTIPNIMFFSTMKRPSREKQDKKIFTEDIDISDISGKPGPPPMGFHSPLIKHPEV |
| | | KSAIEGIKYIAETMKSDQESNNAAAEWKYVAMVMDHILLGVFMLVCIIGTLAVFAGR |
| | | LIELNQQG |
| | SEQ ID NO: 54 | SEHETRLVAKLFKDYSSVVRPVEDHRQVVEVTVGLQLIQLINVDEVNQIVTTNVRL |
| | | KQGDMVDLPRPSCVTLGVPLFSHLQNEQWVDYNLKWNPDDYGGVKKIHIPSEKI |
| | | WRPDLVLYNNADGDFAIVKFTKVLLQYTGHITWTPPAIFKSYCEIIVTHFPFDEQN |
| | | CSMKLGTWTYDGSWVAINPESDQPDLSNFMESGEWVIKESRGWKHSVTYSCCP |
| | | DTPYLDITYHFVMQRL |
| | SEQ ID NO: 55 | NLKWNPDDYGGVKKIHIPSEKIWRPDLVLYNNADGDFAIVKFTKVLLQYTGHITWT |
| | | PPAIFKSYCEIIVTHFPFDEQNCSMKLGTWTYDGSVVAINPES |
| | SEQ ID NO: 56 | LLQYTGHITWTPPAIFKSYCEIIVTHFPFDEQNCSMKLGTWTYDGSWVAINPESD |
| | SEQ ID NO: 57 | MKLGTWTYDGSVVAINPESD |
| Peripherin | SEQ ID NO: 58 | MSHHPSGLRAGFSSTSYRRTFGPPPSLSPGAFSYSSSSRFSSSRLLGSASPSSS |
| derived | | VRLGSFRSPRAGAGALLRLPSERLDFSMAEALNQEFLATRSNEKQELQELNDRF |
| peptides | | ANFIEKVRFLEQQNAALRGELSQARGQEPARADQLCQQELRELRRELELLGRER |
| | | DRVQVERDGLAEDLAALKQRLEEETRKREDAEHNLVLFRKDVDDATLSRLELER |
| | | KIESLMDEIEFLKKLHEEELRDLQVSVESQQVQQVEVEATVKPELTAALRDIRAQY |
| | | ESIAAKNLQEAEEWYKSKYADLSDAANRNHEALRQAKQEMNESRRQIQSLTCEV |
| | | DGLRGTNEALLRQLRELEEQFALEAGGYQAGAARLEEELRQLKEEMARHLREYQ |
| | | ELLNVKMALDIEIATYRKLLEGEESRISVPVHSFASLNIKTTVPEVEPPQDSHSRKT |
| | | VLIKTIETRNGEVVTESQKEQRSELDKSSAHSY |
| | SEQ ID NO: 59 | DSHSRKTVLIKTIETRNGEVVTESQKEQRSELDKSSAHSY |
| | SEQ ID NO: 60 | DSHSKKMVLIRTIETRDGEKVVTESQKEQHSDLDKSSIHSY |
| Aquaporin 4 | SEQ ID NO: 61 | MSDRPTARRWGKCGPLCTRENIMVAFKGVWTQAFWKAVTAEFLAMLIFVLLSLG |
| derived | | STINWGGTEKPLPVDMVLISLCFGLSIATMVQCFGHISGGHINPAVTVAMVCTRKI |
| peptides | | SIAKSVFYIAAQCLGAIIGAGILYLVTPPSVVGGLGVTMVHGNLTAGHGLLVELIITF |
| | | QLVFTIFASCDSKRTDVTGSIALAIGFSVAIGHLFAINYTGASMNPARSFGPAVIMG |
| | | NWENHWIYWVGPIIGAVLAGGLYEYVFCPDVEFKRRFKEAFSKAAQQTKGSYME |
| | | VEDNRSQVETDDLILKPGWHVIDVDRGEEKKGKDQSGEVLSSV |
| | SEQ ID NO: 62 | LVTPPSVVGGLGVTMVHGNLT |
| | SEQ ID NO: 63 | LVTPPSVVGGLGVTMVHGNLTAGHG |
| | SEQ ID NO: 64 | LVTPPSVVGGLGVTMVHGNLTAGHGLLVELIITFQLVFTIFA |
| | SEQ ID NO: 65 | VFYIAAQCLGAIIGAGILYLVTPPSWGGLGVTMVHGNLT |
| ADAMST13 | SEQ ID NO: 66 | MHQRHPRARCPPLCVAGILACGFLLGCWGPSHFQQSCLQALEPQAVSSYLSPG |
| derived | | APLKGRPPSPGFQRQRQRQRRAAGGILHLELLVAVGPDVFQAHQEDTERYVLTN |
| peptides | | LNIGAELLRDPSLGAQFRVHLVKMVILTEPEGAPNITANLTSSLLSVCGWSQTINP |
| | | EDDTDPGHADLVLYITRFDLELPDGNRQVRGVTQLGGACSPTWSCLITEDTGFDL |
| | | GVTIAHEIGHSFGLEHDGAPGSGCGPSGHVMASDGAAPRAGLAWSPCSRRQLL |
| | | SLLSAGRARCVWDPPRPQPGSAGHPPDAQPGLYYSANEQCRVAFGPKAVACTF |
| | | AREHLDMCQALSCHTDPLDQSSCSRLLVPLLDGTECGVEKWCSKGRCRSLVEL |
| | | TPIAAVHGRWSSWGPRSPCSRSCGGGVVTRRRQCNNPRPAFGGRACVGADLQ |
| | | AEMCNTQACEKTQLEFMSQQCARTDGQPLRSSPGGASFYHWGAAVPHSQGDA |
| | | LCRHMCRAIGESFIMKRGDSFLDGTRCMPSGPREDGTLSLCVSGSCRTFGCDG |
| | | RMDSQQVWDRCQVCGGDNSTCSPRKGSFTAGRAREYVTFLTVTPNLTSVYIAN |
| | | HRPLFTHLAVRIGGRYVVAGKMSISPNTTYPSLLEDGRVEYRVALTEDRLPRLEEI |
| | | RIWGPLQEDADIQVYRRYGEEYGNLTRPDITFTYFQPKPRQAWVWAAVRGPCSV |
| | | SCGAGLRWVNYSCLDQARKELVETVQCQGSQQPPAWPEACVLEPCPPYWAVG |
| | | DFGPCSASCGGGLRERPVRCVEAQGSLLKTLPPARCRAGAQQPAVALETCNPQ |
| | | PCPARWEVSEPSSCTSAGGAGLALENETCVPGADGLEAPVTEGPGSVDEKLPA |
| | | PEPCVGMSCPPGWGHLDATSAGEKAPSPWGSIRTGAQAAHVWTPAAGSCSVS |
| | | CGRGLMELRFLCMDSALRVPVQEELCGLASKPGSRREVCQAVPCPARWQYKLA |
| | | ACSVSCGRGWRRILYCARAHGEDDGEEILLDTQCQGLPRPEPQEACSLEPCPP |
| | | RWKVMSLGPCSASCGLGTARRSVACVQLDQGQDVEVDEAACAALVRPEASVP |
| | | CLIADCTYRWHVGTWMECSVSCGDGIQRRRDTCLGPQAQAPVPADFCQHLPKP |
| | | VTVRGCWAGPCVGQGTPSLVPHEEAAAPGRTTATPAGASLEWSQARGLLFSPA |
| | | PQPRRLLPGPQENSVQSSACGRQHLEPTGTIDMRGPGQADCAVAIGRPLGEVV |
| | | TLRVLESSLNCSAGDMLLLWGRLTWRKMCRKLLDMTFSSKTNTLVVRQRCGRP |
| | | GGGVLLRYGSQLAPETFYRECDMQLFGPWGEIVSPSLSPATSNAGGCRLFINVA |
| | | PHARIAIHALATNMGAGTEGANASYILIRDTHSLRTTAFHGQQVLYWESESSQAE |
| | | MEFSEGFLKAQASLRGQYWTLQSWVPEMQDPQSWKGKEGT |
| | SEQ ID NO: 67 | SPRKGSFTAGRAREYVTFLTVTPNLTSVYIANHRPLFTHLAVRIGGRYVVAGKMSI |
| | | SPNTTYPSLLEDGRVEYRVALTEDRLPRLEEIRIWGPLQEDADIQVYRRYGEEYG |
| | | NLTRPDITFTYFQPKPRQA |
| | SEQ ID NO: 68 | CGRQHLEPTGTIDMRGPGQADCAVAIGRPLGEVVTLRVLESSLNCSAGDMLLLW |
| | | GRLTWRKMCRKLLDMTFSSKTNTLVVRQRCGRPGGGVLLRYGSQLAPETFYRE |
| | | CDMQLFGPWGEIVSPSLSPATSNAGGCRLFINVAPHARIAIHALATNMGAGTEGA |
| | | NASYILIRDTHSLRTTAFHGQQVLYWESESSQAEMEFSEGFLKAQASLRGQYWT |
| | | LQSWVPEMQDPQSWKGKEGT |
| | SEQ ID NO: 69 | CGRQHLEPTGTIDMRGPGQADCAVAIGRPLGEVVTLRVLESSLNCSAGDMLLLW |
| | | GRLTWRKMCRKLLDMTFSSKTNTLVVRQRCGRPGGGVLLRYGSQLAPETFYRE |
| | SEQ ID NO: 70 | EIVSPSLSPATSNAGGCRLFINVAPHARIAIHALATNMGAGTEGANASYILIRDTHS |
| | | LRTTAFHGQQVLYWESESSQAEMEFSEGFLKAQASLRGQYWTLQSWVPEMQD |
| | | PQSWKGKEGT |
| | SEQ ID NO: 71 | IGAELLRDPSLGAQFR |
| | SEQ ID NO: 72 | GPSGHVMASDGAAPRAGLA |
| | SEQ ID NO: 73 | SPGGASFYHWGAAVPHSQG |
| | SEQ ID NO: 74 | KRGDSFLDGTRCMPSGPR |
| | SEQ ID NO: 75 | WVNYSCLDQARKE |
| | SEQ ID NO: 76 | ARKELVETVQCQGSQQPPA |
| | SEQ ID NO: 77 | GSLLKTLPPARCRAG |
| | SEQ ID NO: 78 | WRRILYCARAHGED |
| | SEQ ID NO: 79 | RAHGEDDGEEILLDTQCQGLPRPEPQE |
| | SEQ ID NO: 80 | TARRSVACVQLDQGQDVE |
| | SEQ ID NO: 81 | RGPGQADCAVAIGRPLG |
| | SEQ ID NO: 82 | LDMTFSSKTNTLVVRQRCGRPGGGVL |
| | SEQ ID NO: 83 | RPGGGVLLLYGSQLAPET |
| | SEQ ID NO: 84 | GCRLFINVAPHARIAIHA |
| | SEQ ID NO: 85 | ARIAIHALATNMGAGTE |
| | SEQ ID NO: 86 | ASYILIRDTHSLRTTAFHG |
| | SEQ ID NO: 87 | ILYCARAHG |
| | SEQ ID NO: 88 | GCRLFINVAPHARIA |
| | SEQ ID NO: 89 | FINVAPHAR |
| | SEQ ID NO: 90 | RRSVACVQLDQGQD |
| | SEQ ID NO: 91 | VQLDQGQDV |
| | SEQ ID NO: 92 | GPGQADCAVAIGRPL |
| | SEQ ID NO: 93 | CAVAIGRPL |
| | SEQ ID NO: 94 | RPGGGVLLLYGSQLAPE |
| | SEQ ID NO: 95 | LLRYGSQLA |
| | SEQ ID NO: 96 | ARIAIHALATNMGAGT |
| | SEQ ID NO: 97 | RIAIHALATNMGAGT |
| | SEQ ID NO: 98 | RIAIHALATNMGAG |
| | SEQ ID NO: 99 | IAIHALATNMGAGT |
| | SEQ ID NO: 100 | IHALATNMG |
| | SEQ ID NO: 101 | LDMTFSSKTNTLVVR |
| | SEQ ID NO: 102 | FSSKTNTLV |
| | SEQ ID NO: 103 | ARRSVACCQLDQGQDVE |
| | SEQ ID NO: 104 | LKTLPPARC |
| | SEQ ID NO: 105 | IGAELLRDPSLGAQF |
| | SEQ ID NO: 106 | IGAELLRDPSLGAQ |
| | SEQ ID NO: 107 | IGAELLRD |
| | SEQ ID NO: 108 | LLRDPSLGA |
| | SEQ ID NO: 109 | RRSVACVQLDQGQDVE |
| | SEQ ID NO: 110 | RLFINVAPHARIA |
| | SEQ ID NO: 111 | RLFINVAPHAR |
| | SEQ ID NO: 112 | GPSGHVMASDGAAPRAG |
| | SEQ ID NO: 113 | MASDGAAPR |
| | SEQ ID NO: 114 | RAHGEDDGEEILLDTQCQGLPRPEPQ |
| | SEQ ID NO: 115 | DGEEILLDTQCQGLPR |
| | SEQ ID NO: 116 | EEILLDTQCQGLPR |
| | SEQ ID NO: 117 | ILLDTQCQG |
| | SEQ ID NO: 118 | TNTLWRQRCGRPGGGVL |
| | SEQ ID NO: 119 | LWVRQRCGR |
| | SEQ ID NO: 120 | LFINVAPHARIAIHA |
| | SEQ ID NO: 121 | FINVAPHARIAIHA |
| | SEQ ID NO: 122 | INVAPHARIAIHA |
| | SEQ ID NO: 123 | ASYILIRDTHSLRTTAFH |
| | SEQ ID NO: 124 | ASYILIRDTHSLRTTAF |
| | SEQ ID NO: 125 | LIRDTHSLR |
| | SEQ ID NO: 126 | GDSFLDGTRCMPSGPR |
| | SEQ ID NO: 127 | LDGTRCMPS |
| | SEQ ID NO: 128 | QADCAVAIG |
| | SEQ ID NO: 129 | LRDPSLGAQ |
| | SEQ ID NO: 130 | WGAAVPHSQ |
| | SEQ ID NO: 131 | NYSCLDQAR |
| | SEQ ID NO: 132 | RKELVETVQCQGSQQPPA |
| | SEQ ID NO: 133 | KELVETVQCQGSQQPP |
| | SEQ ID NO: 134 | ELVETVQCQ |
| | SEQ ID NO: 135 | ASYILIRDTHSLRTTA |
| | SEQ ID NO: 136 | SYILIRDTHSLRTTA |
| | SEQ ID NO: 137 | ASYILIRDT |

X represents citrulline

In another embodiment, the antigen comprised in the composition of the first aspect is an allergen. “Allergens” are any substances that can cause an undesired (e.g., a Type 1 hypersensitive) immune response (i.e., an allergic response or reaction) in a subject. Allergens include, but are not limited to, plant allergens (e.g., pollen, ragweed allergen), insect allergens, insect sting allergens (e.g., bee sting allergens), animal allergens (e.g., pet allergens, such as animal dander or cat Fel d 1 antigen), latex allergens, mold allergens, fungal allergens, cosmetic allergens, drug allergens, food allergens, dust, insect venom, viruses, bacteria, etc. Food allergens include, but are not limited to milk allergens, egg allergens, nut allergens (e.g., peanut or tree nut allergens, etc. (e.g., walnuts, cashews, etc.)), fish allergens, shellfish allergens, soy allergens, legume allergens, seed allergens and wheat allergens. Insect sting allergens include allergens that are or are associated with bee stings, wasp stings, hornet stings, yellow jacket stings, etc. Insect allergens also include house dust mite allergens (e.g., Der P1 antigen) and cockroach allergens. Drug allergens include allergens that are or are associated with antibiotics, NSAIDs, anaesthetics, etc. Pollen allergens include grass allergens, tree allergens, weed allergens, flower allergens, etc.

In another embodiment, the antigen comprised in the composition of the first aspect is associated with organ or tissue rejection. Examples of such antigens include, but are not limited to, antigens from allogeneic cells, e.g., antigens from an allogeneic cell extract and antigens from other cells, such as endothelial cell antigens. Antigens also include those associated with a transplantable graft. Such antigens are associated with a transplantable graft, or an undesired immune response in a recipient of a transplantable graft that is generated as a result of the introduction of the transplantable graft in the recipient, that can be presented for recognition by cells of the immune system and that can generate an undesired immune response. Transplant antigens include those associated with organ or tissue rejection or graft versus host disease. Transplant antigens may be obtained or derived from cells of a biological material or from information related to a transplantable graft. Transplant antigens generally include proteins, polypeptides, peptides, lipoproteins, glycolipids, polynucleotides or are contained or expressed in cells. Information related to a transplantable graft is any information about a transplantable graft that can be used to obtain or derive transplant antigens. Such information includes information about antigens that would be expected to be present in or on cells of a transplantable graft such as, for example, sequence information, types or classes of antigens and/or their MHC Class I, MHC Class II or B cell presentation restrictions. Such information may also include information about the type of transplantable graft (e.g., autograft, allograft, xenograft), the molecular and cellular composition of the graft, the bodily location from which the graft is derived or to which the graft is to be transplanted (e.g., whole or partial organ, skin, bone, nerves, tendon, neurons, blood vessels, fat, cornea, etc.).

In another embodiment, the antigen comprised in the composition of the first aspect is a therapeutically active agent (herein also referred to as “drug”) that can generate an undesired immune response. Said therapeutically active agent can be, for example, a therapeutic protein. Therapeutic protein antigens generally include proteins, polypeptides, peptides, lipoproteins, or are contained or expressed in, by or on cells. Therapeutic proteins include, but are not limited to, infusible therapeutic proteins, enzymes, enzyme cofactors, hormones, blood clotting factors, cytokines and interferons, growth factors, monoclonal antibodies, and polyclonal antibodies (e.g., that are administered to a subject as a replacement therapy), and proteins associated with Pompe's disease (e.g., alglucosidase alfa, rhGAA (e.g., Myozyme and Lumizyme (Genzyme). Therapeutic proteins also include proteins involved in the blood coagulation cascade. Therapeutic proteins include, but are not limited to, Factor VIII, Factor VII, Factor IX, Factor V, von Willebrand Factor, von Heldebrant Factor, tissue plasminogen activator, insulin, growth hormone, erythropoietin alfa, VEGF, thrombopoietin, lysozyme, antithrombin and the like. Therapeutic proteins also include adipokines, such as leptin and adiponectin. Other examples of therapeutic proteins are as described below. Also contemplated as antigens in the composition of the first aspect are fragments or derivatives of any of the therapeutic proteins disclosed herein provided as the antigen.

Examples of therapeutic proteins used in enzyme replacement therapy of subjects having a lysosomal storage disorder include, but are not limited to, imiglucerase for the treatment of Gaucher's disease (e.g., CEREZYME™), a-galactosidase A (a-gal A) for the treatment of Fabry disease (e.g., agalsidase beta, FABRYZYME™), acid a-glucosidase (GAA) for the treatment of Pompe disease (e.g., alglucosidase alfa, LUMIZYME™, MYOZYME™), arylsulfatase B for the treatment of Mucopolysaccharidoses (e.g., laronidase, ALDURAZYME™, idursulfase, ELAPRASE™, arylsulfatase B, NAGLAZYME™). Other examples of enzymes include oxidoreductases, transferases, hydrolases, lyases, isomerases, and ligases.

Examples of therapeutic proteins are also hormones includen Melatonin (N-acetyl-5-methoxytryptamine), Serotonin, Thyroxine (or tetraiodothyronine) (a thyroid hormone), Triiodothyronine (a thyroid hormone), Epinephrine (or adrenaline), Norepinephrine (or noradrenaline), Dopamine (or prolactin inhibiting hormone), Antimullerianormone (or mullerian inhibiting factor or hormone), Adiponectin, Adrenocorticotropic hormone (or corticotropin), Angiotensinogen and angiotensin, Antidiuretic hormone (or vasopressin, arginine vasopressin), Atrial-natriuretic peptide (or atriopeptin), Calcitonin, Cholecystokinin, Corticotropin-releasing hormone, Erythropoietin, Follicle-stimulating hormone, Gastrin, Ghrelin, Glucagon, Glucagon-like peptide (GLP-1), GIP, Gonadotropin-releasing hormone, Growth hormone-releasing hormone, Human chorionic gonadotropin, Human placental lactogen, Growth hormone, Inhibin, Insulin, Insulin-like growth factor (or somatomedin), Leptin, Luteinizing hormone, Melanocyte stimulating hormone, Orexin, Oxytocin, Parathyroid hormone, Prolactin, Relaxin, Secretin, Somatostatin, Thrombopoietin, Thyroid-stimulating hormone (or thyrotropin), Thyrotropin-releasing hormone, Cortisol, Aldosterone, Testosterone, Dehydroepiandrosterone, Androstenedione, Dihydrotestosterone, Estradiol, Estrone, Estriol, Progesterone, Calcitriol (1,25-dihydroxyvitamin D3), Calcidiol (25-hydroxyvitamin D3), Prostaglandins, Leukotrienes, Prostacyclin, Thromboxane, Prolactin releasing hormone, Lipotropin, Brain natriuretic peptide, Neuropeptide Y, Histamine, Endothelin, Pancreatic polypeptide, Renin, and Enkephalin.

Examples blood and blood coagulation factors include Factor I (fibrinogen), Factor II (prothrombin), tissue factor, Factor V (proaccelerin, labile factor), Factor VII (stable factor, proconvertin), Factor VIII (antihemophilic globulin), Factor IX (Christmas factor or plasma thromboplastin component), Factor X (Stuart-Prower factor), Factor Xa, Factor XI, Factor XII (Hageman factor), Factor XIII (fibrin-stabilizing factor), von Willebrand factor, prekallikrein (Fletcher factor), high-molecular weight kininogen (HMWK) (Fitzgerald factor), fibronectin, fibrin, thrombin, antithrombin III, heparin cofactor II, protein C, protein S, protein Z, protein Z-related protease inhibitot (ZPI), plasminogen, alpha 2-antiplasmin, tissue plasminogen activator (tPA), urokinase, plasminogen activator inhibitor-1 (PAI1), plasminogen activator inhibitor-2 (PAI2), cancer procoagulant, and epoetin alfa (Epogen, Procrit).

Examples of cytokines include lymphokines, interleukins, and chemokines, type 1 cytokines, such as IFN-γ, TGF-β, and type 2 cytokines, such as IL-4, IL-10, and IL-13.

Examples of growth factors include Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-β), Tumour_necrosis_factor-alpha (TNF-α), Vascular endothelial growth factor (VEGF), Wnt Signaling Pathway, placental growth factor (P1GF), [(Foetal Bovine Somatotrophin)] (FBS), IL-1, IL-2, IL-3, IL-4, IL-5, IL-6, and IL-7.

Examples of monoclonal antibodies include Abagovomab, Abciximab, Adalimumab, Adecatumumab, Afelimomab, Afutuzumab, Alacizumab pegol, ALD, Alemtuzumab, Altumomab pentetate, Anatumomab mafenatox, Anrukinzumab, Anti-thymocyte globin, Apolizumab, Arcitumomab, Aselizumab, Atlizumab (tocilizumab), Atorolimumab, Bapineuzumab, Basiliximab, Bavituximab, Bectumomab, Belimumab, Benralizumab, Bertilimumab, Besilesomab, Bevacizumab, Biciromab, Bivatuzumab mertansine, Blinatumomab, Brentuximab vedotin, Briakinumab, Canakinumab, Cantuzumab mertansine, Capromab pendetide, Catumaxomab, Cedelizumab, Certolizumab pegol, Cetuximab, Citatuzumab bogatox, Cixutumumab, Clenoliximab, Clivatuzumab tetraxetan, Conatumumab, Dacetuzumab, Daclizumab, Daratumumab, Denosumab, Detumomab, Dorlimomab aritox, Dorlixizumab, Ecromeximab, Eculizumab, Edobacomab, Edrecolomab, Efalizumab, Efungumab, Elotuzumab, Elsilimomab, Enlimomab pegol, Epitumomab cituxetan, Epratuzumab, Erlizumab, Ertumaxomab, Etaracizumab, Exbivirumab, Fanolesomab, Faralimomab, Farletuzumab, Felvizumab, Fezakinumab, Figitumumab, Fontolizumab, Foravirumab, Fresolimumab, Galiximab, Gantenerumab, Gavilimomab, Gemtuzumab ozogamicin, GCI008, Girentuximab, Glembatumumab vedotin, Golimumab, Gomiliximab, Ibalizumab, Ibritumomab tiuxetan, Igovomab, Imciromab, Infliximab, Intetumumab, Inolimomab, Inotuzumab ozogamicin, Ipilimumab, Iratumumab, Keliximab, Labetuzumab, Lebrikizumab, Lemalesomab, Lerdelimumab, Lexatumumab, Libivirumab, Lintuzumab, Lorvotuzumab mertansine, Lucatumumab, Lumiliximab, Mapatumumab, Maslimomab, Matuzumab, Mepolizumab, Metelimumab, Milatuzumab, Minretumomab, Mitumomab, Morolimumab, Motavizumab, Muromonab-CD3, Nacolomab tafenatox, Naptumomab estafenatox, Natalizumab, Nebacumab, Necitumumab, Nerelimomab, Nimotuzumab, Nofetumomab merpentan, Ocrelizumab, Odulimomab, Ofatumumab, Olaratumab, Omalizumab, Oportuzumab monatox, Oregovomab, Otelixizumab, Pagibaximab, Palivizumab, Panitumumab, Panobacumab, Pascolizumab, Pemtumomab, Pertuzumab, Pexelizumab, Pintumomab, Priliximab, Pritumumab, Rafivirumab, Ramucirumab, Ranibizumab, Raxibacumab, Regavirumab Reslizumab, Rilotumumab, Rituximab, Robatumumab, Rontalizumab, Rovelizumab, Ruplizumab, Satumomab pendetide, Sevirumab, Sibrotuzumab, Sifalimumab, Siltuximab, Siplizumab, Solanezumab, Sonepcizumab, Sontuzumab, Stamulumab, Sulesomab, Tacatuzumab tetraxetan, Tadocizumab, Talizumab, Tanezumab, Taplitumomab paptox, Tefibazumab, Telimomab aritox, Tenatumomab, Teneliximab, Teplizumab, Ticilimumab (tremelimumab), Tigatuzumab, Tocilizumab (atlizumab), Toralizumab, Tositumomab, Trastuzumab, Tremelimumab, Tucotuzumab celmoleukin, Tuvirumab, Urtoxazumab, Ustekinumab, Vapaliximab, Vedolizumab, Veltuzumab, Vepalimomab, Visilizumab, Volociximab, Votumumab, Zalutumumab, Zanolimumab, Ziralimumab, and Zolimomab aritox.

Examples of infusion therapy or injectable therapeutic proteins include, for example, Tocilizumab (Roche/Actemra®), alpha-1 antitrypsin (Kamada/AAT), Hematide® (Affymax and Takeda, synthetic peptide), albinterferon alfa-2b (Novartis/Zalbin™) Rhucin® (Pharming Group, C1 inhibitor replacement therapy), tesamorelin (Theratechnologies/Egrifta, synthetic growth hormone-releasing factor), ocrelizumab (Genentech, Roche and Biogen), belimumab (GlaxoSmithKline/Benlysta®), pegloticase (Savient Pharmaceuticals/Krystexxa™), taliglucerase alfa (Protalix/Uplyso), agalsidase alfa (Shire/Replagal®), velaglucerase alfa (Shire). Additional therapeutic proteins useful in accordance to aspects of this invention will be apparent to those of skill in the art, and the invention is not limited in this respect.

In particular embodiments, the antigen comprised in the liposomal composition of the first aspect is a viral antigen, in particular, a viral vector, for example, a viral transfer vector. Viral vectors are used to transfer therapeutic polynucleotides into the cells and are increasingly being employed in gene therapy, as well as in RNA or DNA-based vaccines. Unfortunately, however, redosing of gene therapy is limited by immunogenicity which often causes severe toxicities. Enhancing tolerance to these gene vectors is therefore of great interest and may be achieved by using the liposomal compositions of the present disclosure. Non-limiting viral vectors contemplated in the sense of the present description are retroviral vectors, lentiviral vectors, herpes simplex virus (HSV)-based vectors, adenovirus-based vectors, adeno-associated virus (AAV)-based vectors, and AAV-adenoviral chimeric vectors. Adenoviral vectors may be selected from the group consisting of subgroup A, subgroup B, subgroup C, subgroup D, subgroup E, and subgroup F adenoviral vectors. Lentiviral vectors may be selected from the group consisting of HIV, SIV, FIV, EIAV, and ovine lentiviral vectors. Adeno-associated viral vectors may be selected from the group consisting of AAV1, AAV2, AAV5, AAV6, AAV6.2, AAV7, AAV8, AAV9, AAV10, and AAVI I adeno-associated viral vector. In some embodiments the viral vector is a chimeric viral vector, for example, an AAV-adenoviral vector. In particular embodiments, the antigen is a viral component, in particular, a viral protein. In one particular embodiment the antigen is an envelope protein. In very particular embodiments, the antigen is a viral capsid component, for example a viral capsid protein, such as capsids proteins VP1, VP2, and VP3. In a preferred embodiment, when the antigen is a viral antigen, said viral antigen is selected from the group consisting of capsid proteins VP1, VP2, VP3, and combinations thereof.

As would be apparent to the skilled person, immunogenic fragments of any of the above antigenic proteins are also contemplated as antigens in the sense of the present disclosure.

It is known that some oxidative and non-oxidative post-translational modifications can generate neoantigens from original antigens. Thus, in one embodiment, the antigen is a neoantigen. Examples of non-oxidative and oxidative post-translational modifications that may give rise to neoantigens are citrullination, glycosylation, sumoylation, neddylation, deamination, deamidation, hydroxylation, sulfation, oxidation, carbamylation, pegylation, succinylation, alkylation, sialylation phosphorylation, ubiquitination, nitrosylation, methylation, acetylation, arginylation, amidation, tyrosination, lipidation. In other embodiments the antigen may be a post-translational modified antigen. Thus, in another embodiment, the antigen is a post-translational modified antigen of any of the previous embodiments.

In further embodiments, the antigens in the liposome compositions of the first aspect are provided in the form of a nucleic acid that encodes the peptide, polypeptide or protein. The nucleic acid may be DNA or RNA, such as mRNA. In embodiments, the nucleic acid encoding the antigenic polypeptide is comprised within an expression vector that can be transcribed when transfected into a cell. In some embodiments, the expression vector may comprise a plasmid, viral particle, retrovirus, or adenovirus, amongst others. For example, the antigen may be a polynucleotide encoding for a viral antigen, such as a viral capsid antigenic protein, for example viral capsid proteins VP1, VP2 or VP3.

### Preparation of the Liposome Composition

Various methodologies well-known to those skilled in the art can be used to prepare the liposome composition of the first aspect of the disclosure.

Liposomes carrying an antigen may be formed by directly entrapping the autoantigen during liposome formation by well-known methods, such as lipid thin film hydration method and solvent injection method. In one embodiment, the liposomes are prepared by the thin film hydration method (Bangham et al., J. Mol. Biol., 13, 238 (1965). In another embodiment, the liposomes are prepared by solvent injection method (Pons et al. International Journal of Pharmaceutics 95 (1993) 51-56).

In one embodiment, the liposomes carrying an antigen may be prepared by a process comprising: (a) preparing a lipid blend in an appropriate solvent, e.g. chloroform, (b) removing the solvent, e.g., by evaporation under vacuum, (c) hydrating the lipid blend with an appropriate buffer, e.g. phosphate buffer saline, containing an antigen to obtain the antigen-containing liposomes.

In another embodiment, the liposomes carrying an antigen may be prepared by a process comprising: (a) preparing a lipid blend in an appropriate solvent, e.g. ethanol, and (b) injecting said lipid blend in a solution containing at least one antigen and an appropriate buffer, e.g. phosphate buffer saline, to obtain the antigen-containing liposomes.

When the liposome carries more than one antigen, the hydrating step (c) or the injection step (b) are performed in the presence of a buffer containing a mixture of said antigens in the desired proportion. Such proportion takes into account the particular encapsulating efficiency for each antigen.

In some embodiments, the weight ratio between the total amount of lipid forming the liposome membrane vs total amount of antigen(s) is comprised from 1000:1 to 1:1, or from 500:1 to 2:1, or from 350:1 to 10:1, or from 250:1 to 15:1, or from 210:1 to 20:1, or from 190:1 to 30:1. In another a particular embodiment, the weight ratio between the total amount of lipid forming the liposome membrane vs total amount of antigen(s) is comprised from 350:1 to 10:1. In a still more particular embodiment, the weight ratio between the total amount of lipid forming the liposome membrane vs total amount of antigen(s) is comprised from 250:1 to 20:1. In still more particular embodiment, the weight ratio between the total amount of lipid forming the liposome membrane vs total amount of antigen(s) is comprised from 210:1 to 25:1. In still more particular embodiment, the weight ratio between the total amount of lipid forming the liposome membrane vs total amount of antigen(s) is comprised from 190:1 to 30:1.

Other methods known in the art may also be used for obtaining the liposomes carrying an antigen. For instance, some embodiments contemplate first obtaining the liposomes and then including the antigen. There are well-known methods in the state of the art to include a compound within a liposome (see Maurer N. et al., Expert Opin Biol Ther, 2001, vol. 1(6), p. 923-47; Waterhouse D. N. et al., Methods Enzymol., 2005; vol. 391, p. 40-57; Urbán P. et al., Nanosc. Res. Lett., 2011, vol. 6, p. 620).

The resulting antigen-containing liposomes obtained as described above, or in any other way known to the skilled person, may be subjected to further purification, homogenization and/or separation steps. In most embodiments, a purification step is applied to remove the non-encapsulated peptide. Said purification step may be performed, e.g. by centrifugation, filtration, tangential flow filtration, dialysis, gel-permeation chromatography, ion-exchange chromatography, size exclusion chromatography. etc. For example, purification step to remove non-encapsulated antigen may be performed by filtering through a 100 KDa filter. The antigen-containing liposomes may be further homogenized or separated by size. Extrusion can be used to size-homogenize liposomes, i.e., to produce liposomes having a predetermined mean size by forcing the liposomes, under pressure, through filters with a specific pore. Filtration, e.g., tangential flow filtration, can also be used to purify and separate the liposomes according to their size, i.e., to produce a population of liposomes having fewer impurities and a desired size distribution. Other methods that may separate liposome populations according to their size are centrifugation (e.g., ultracentrifugation), size exclusion chromatography, gel-permeation chromatography, and combinations thereof.

In one embodiment, the liposome composition of the first aspect is prepared by a process comprising: obtaining liposomes carrying an antigen by any of the methods described above and, optionally, further purification, separation and/or enrichment steps. In a particular embodiment, the process comprises a purification step to remove the non-encapsulated peptide. In another embodiment, the process further comprises an homogenization step. In another embodiment, the process further comprises a separation step. In some embodiments, the process further comprises an enrichment step. For example, one part of the composition obtained by any of the methods described above may be subjected to separation in order to separate a liposome population, for example, the liposome population of size below 200 nm. Said separate liposome population may then be added to a composition comprising the two populations, whereby the selected liposome population, for example, the liposome population of size below 200 nm, is consequently enriched. In other embodiments, extrusion, physical separation or sonication are used to size-homogenize liposomes or to obtain desired liposome populations. The homogenized liposomes or separate liposome populations may then be added to a liposome composition comprising the populations of interest as defined in the first aspect, whereby the composition is enriched in a particular liposome population.

The composition of the first aspect of the disclosure may be obtained by separately preparing each of the two liposome populations as defined in the first aspect and then mixing them in an appropriate proportion. The composition may also be obtained by a method that yields a composition already comprising the two populations as defined in the first aspect.

In one embodiment, the liposome composition of the first aspect may be prepared by a process comprising: (a) obtaining liposomes carrying an antigen, e.g. by any of the methods described above, (b) separating the liposomes of size 2-200 nm, (c) separating the liposomes of size 500-2000 nm, and (d) mixing the liposomes of size 2-200 nm with the liposomes of size 500-2000 nm.

In another embodiment, the liposome composition of the first aspect is prepared by a process comprising: obtaining liposomes carrying an antigen, e.g. by any of the methods described above, (b) subjecting part of the liposome composition obtained in (a) to a separation step to separate liposomes of size 2-200 nm, (c) adding the separated liposomes of size 2-200 nm to the liposome composition obtained in (a) to obtain a liposome composition enriched in liposomes of size 2-200 nm.

The present disclosure contemplates a liposome composition obtainable by any of the above methods.

In a particular embodiment, a liposome composition with a wide size polydispersity and carrying an antigen may be prepared by the solvent injection method in a process comprising the preparation of a lipid blend in an appropriate solvent, e.g. ethanol, and injecting said lipid blend in a solution containing at least one antigen and an appropriate buffer, e.g. phosphate buffer saline, to obtain the antigen-containing liposomes. Low injection speed, specific number of injections and gentle shaking instead of vortexing or high-speed stirring facilitates the formation of larger size liposomes. By not applying any homogenization step afterwards, e.g. extrusion under pressure and not applying any further purification step to remove smaller liposomes, e.g. tangential flow filtration, the liposomal composition will present a wide size distribution. To reduce the mean diameter of the liposome composition, several consecutive extrusion cycles can be applied to the heterogeneous and polydisperse composition. To accomplish this, liposomes are sequentially filtered through a series of polycarbonate membranes with pores of decreasing diameter, thus conveniently and reproducibly obtaining liposomes with a mean size approaching the membrane pore. Furthermore, applying to the formulation a brief sonication treatment, directly with a tip or indirectly in a bath sonicator at room temperature, also reduces the liposome mean diameter. Other industrial homogenization methods to break down the large liposomes are micro fluidization, high-pressure homogenization, and shear force-induced homogenization techniques. In one embodiment the disclosure also contemplates a liposome composition obtainable by this particular method.

### Formulations

In one embodiment, the liposome composition of the first aspect is a pharmaceutical composition. The disclosure thus provides a pharmaceutical or veterinary composition comprising a therapeutically effective amount of the liposome composition as defined in the first aspect, optionally together with other appropriate pharmaceutically or veterinary acceptable excipients or carriers. In another embodiment, the disclosure provides a pharmaceutical or veterinary composition consisting essentially of a therapeutically effective amount of the liposome composition as defined in the first aspect and appropriate pharmaceutically or veterinary acceptable excipients or carriers. In another embodiment, the disclosure provides a pharmaceutical or veterinary composition consisting of a therapeutically effective amount of the liposome composition as defined in the first aspect and appropriate pharmaceutically or veterinary acceptable excipients or carriers.

The expression “therapeutically effective amount” as used herein, refers to the amount of liposomes, in particular, of liposomes of the first and second populations according to the composition of the first aspect, that, when administered, is sufficient to prevent development of, or alleviate to some extent, one or more of the symptoms of the disorder which is addressed. The particular dose of compound administered according to this disclosure will of course be determined by the particular circumstances surrounding the case, including the antigen administered, the route of administration, the particular condition being treated, and the similar considerations. In one embodiment, therapeutically effective amounts in the sense of the present disclosure involve reducing the level of an undesired immune response. In other embodiments, therapeutically effective amounts involve preventing an undesired immune response altogether. In other embodiments, therapeutically effective amounts involve delaying the occurrence of an undesired immune response. A therapeutically effective amount in the sense of the present disclosure result in a tolerogenic immune response in a subject to an antigen. The achievement of any of the foregoing can be monitored by routine methods.

The present disclosure also contemplates compositions wherein the liposomes carry more than one antigen and compositions comprising different liposomes, each carrying a different antigen. Preferably all antigens contained in the compositions of the first aspect are related to the same immunological disorder.

In the present disclosure, the term “pharmaceutically acceptable excipients or carriers” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, antigenicity or other problems or complications commensurate with a reasonable benefit/risk ratio. Likewise, the term “veterinary acceptable” means suitable for use in contact with a non-human animal.

The antigen-containing liposomes of the present compositions are considered to be the active agent by themselves, thus, the antigen-containing liposomes of particular size range are responsible of promoting tolerance directed to the specifically contained antigen(s). Thus, it is noteworthy that no other active agent is required in order to induce tolerance. Importantly, it is not required that the inventive compositions are used together with other immunomodulatory agents. In particular, it is not required that the compositions contain or are used in combination with immunosuppressants. In particular embodiments, the composition of the first aspect does not contain an immunosuppressant. Otherwise worded, the composition of the first aspect is devoid immunosuppressants.

Nevertheless, even though it is not required to attain the technical effect, the present disclosure also contemplates combining the liposome composition described herein with other active agents. Thus, the pharmaceutical compositions contemplated herein may contain additional active agents, such as other immunomodulatory agents, for example an immunosuppressant. Examples of immunosuppressants are glucocorticoids (prednisolone, methylprednisone, betamethasone), cytostatics (methotrexate . . . ), antibodies (rituximab . . . ), drugs actin on immunophilins (ciclosporin, tacrolimus, sirolimus, everolimus . . . ), interferons, TNF binding proteins, mycophenolate, and small biological agents (fingolimod, myriocin)

The formulation of the compositions of the disclosure greatly depends on the administration route. In one embodiment, the pharmaceutical composition is administered to a patient orally. Oral compositions include tablets, powders, capsules, sachets, as well as liquid syrups, suspensions and elixirs, all of which may be formulated by methods well known in the art. The compositions of the disclosure can also be administered to a patient by intravenous, intraarterial, intraperitoneal (i.p.), subcutaneous, intramuscular or intradermal route. Compositions adequate for these routes of administration are also well known in the art and include solutions for injection, solutions for perfusion, powder for reconstitution of liquid injections, and pre-filled syringes. In the sense of the present disclosure it may also be adequate to formulate the compositions disclosed herein for intranasal or inhaled administration, rectal administration or for topical administration in the form of, for instance, a cream, a gel, an ointment or a dermal patch. Methods for the preparation of these formulations are known in the art. Further, the compositions of the present disclosure can be formulated as a controlled release dosage form. Controlled release dosage forms are known in the art and particularly desirable for the treatment of chronic diseases or for the administration of active agents that can be toxic at high doses or that show a low half-life pattern when administered to the patient.

Herein disclosed is also a kit of parts that comprises:
(a) a liposome composition as defined above, optionally together with pharmaceutically acceptable excipients or carriers;
(b) optionally a further active agent; and
(c) optionally, instructions for its use.

Herein disclosed is also a vessel or injection device which comprises the liposome composition as defined above, preferably together with pharmaceutically acceptable excipients or carriers.

### Therapeutic Uses

As mentioned above, the liposome compositions of the present disclosure may be used in therapy, since they promote tolerance to the antigen(s) contained in the composition. In one embodiment, the inducing of tolerance comprises B-cell mediated tolerance. In another embodiment, the inducing of tolerance comprises T-cell mediated tolerance, in particular, tolerance mediated through tolerogenic presentation of the antigen by dendritic cells. When referring to the medical uses of the liposome compositions of the disclosure, it is contemplated that said compositions may be pharmaceutical compositions comprising a therapeutically effective amount of the liposome composition as defined in the first aspect, optionally together with other appropriate pharmaceutically or veterinary acceptable excipients or carriers.

The liposome composition of the disclosure, as widely explained above, contains two liposome populations of different size, both containing an antigen a having particular membrane composition. These liposome populations constitute by themselves the active ingredient that elicits tolerance and has the effect of treating conditions related to a dysfunctional, e.g. exacerbated, immune response, without the need to add further active ingredients, such as immunosuppressants. Each of the liposome populations target a different mechanism of tolerogenesis. While the liposomal population of size 500 nm or greater elicits antigen-specific tolerance through a mechanism that resembles efferocytosis and involves tolerogenic presentation of the antigen by dendritic cells, the liposomal population of size 2-200 nm is able to induce tolerance through B-cells. The dual effect is previously undisclosed and provides an improved tolerogenic effect. Moreover, the herein disclosed liposome composition, thanks to the dual effect, is effective in treating immune disorders that could not be treated by previous tolerance-inducing agents.

The liposome composition as defined above is for use in immunomodulation, more in particular, for suppressing an excessive immune response to a particular antigen. In some embodiments, the liposome composition as defined above is for use in the treatment of a disorder associated to an abnormal, mostly, excessive, immune response. It is important that the tolerogenic/immunomodulatory effect of the liposome composition is antigen-specific (antigen-specific). Thus, in certain embodiments the liposome composition as defined above is for use in the treatment of a disorder associated to an abnormal, mostly, excessive, immune response, wherein said liposome composition restores tolerance to the antigen comprised in the liposome composition.

In one embodiment, the disorder associated to an abnormal, mostly excessive, immune response is selected from an autoimmune disease, allergy, drug hypersensitivity, and transplant rejection.

In the sense of the present disclosure, the term “treatment” includes a prophylactic treatment before the clinical onset of the symptoms caused by the immune disorder or a therapeutic treatment after the clinical onset of symptoms caused by the immune disorder. In a particular embodiment the treatment is a prophylactic treatment. In one embodiment, the prophylactic treatment comprises partial or total prevention of the immune disorder. In one embodiments, the abnormal immune response to an autoantigen is prevented, whereby the pathogenic events underlying the abnormal immune response are not triggered. In another particular embodiment, the treatment comprises ameliorating, slowing down, arresting or reversing the pathological mechanisms underlying the immune disorder. In particular embodiments the treatment comprises ameliorating, slowing down, arresting, delaying or reversing the clinical symptoms of the immune disorder.

The present disclosure contemplates treatment of the immune condition in patients that, despite having an abnormal immune response and some tissue damage, do not show clinical symptoms or show only few clinical symptoms of the disease. This stage is often called “pre-clinical” stage and is typical of transplant rejections and many autoimmune diseases, for example in T1D, where it is called prediabetes. In pre-diabetes, pancreatic B cells are damaged to some extent but only some of the diagnostic criteria for diabetes are met. The disease at this pre-clinical stage may be effectively treated with the liposome composition of the disclosure. Thus, one embodiment is directed to the treatment of an autoimmune disease during the pre-clinical stage. Additionally, advanced stages of immune disease where tissue damage is high and clinical symptoms are apparent, may also be effectively treated by administering an effective amount of the liposome composition of the disclosure.

In a particular embodiment, the disorder is an autoimmune disease selected from the group consisting of T1D, lupus erythematosus, arthritis (including rheumatoid arthritis, juvenile rheumatoid arthritis, osteoarthritis, reactive arthritis, psoriatic arthritis), multiple sclerosis (MS), neuromyelitis optica, Addison's disease, celiac disease, dermatomyositis, Hashimoto's thyroiditis, myasthenia gravis, pernicious anemia, autoimmune hemolitic anemia, autoimmune neutrophenia, Graves' disease, psoriasis, Sjogren syndrome, including keratoconjunctivitis sicca secondary to Sjogren's Syndrome, autoimmune thyroiditis, dermatitis (including atopic dermatitis and eczematous dermatitis), alopecia areata, Crohn's disease, aphthous ulcer, iritis, conjunctivitis, keratoconjunctivitis, ulcerative colitis, autoimmune uveitis, allergic encephalomyelitis, acute necrotizing hemorrhagic encephalopathy, idiopathic bilateral progressive sensorineural hearing loss, aplastic anemia, pure red cellanemia, idiopathic thrombocytopenia, immune-mediated thrombotic thrombocytopenia purpura, polychondritis, Wegener's granulomatosis, chronic active pepatitis, Stevens Johnson syndrome, idiopathic sprue, lichen planus, sarcoidosis, primary biliary cirrhosis, uveitis posterior, and interstitial lung fibrosis.

In a particular embodiment, the disclosure provides a liposome composition as defined in the first aspect, or pharmaceutical composition thereof, for use in the treatment of MS. In another embodiment, the autoimmune disease to be treated is T1D. In another embodiment, the liposome composition as defined in the first aspect, or pharmaceutical composition thereof, is for use in the treatment of T1D in a prediabetic subject. In another particular embodiment, the autoimmune disease to be treated is myasthenia gravis. In another particular embodiment, the autoimmune disease to be treated is selected from the group consisting of rheumatoid arthritis, neuromyelits optica, myositis or ansysynthetase syndrome, thrombotic thrombocytopenia purpura, and celiac disease.

In another particular embodiment, the disorder is an allergy or allergic condition. “Allergies” or “allergic conditions” include, but are not limited to, allergic asthma, hay fever, hives, eczema, plant allergies, bee sting allergies, pet allergies, latex allergies, mold allergies, cosmetic allergies, food allergies, allergic rhinitis or coryza, topic allergic reactions, anaphylaxis, atopic dermatitis, hypersensitivity reactions and other allergic conditions. In some embodiments, the allergy is a food allergy. Food allergies include, but are not limited to, milk allergies, egg allergies, nut allergies, fish allergies, shellfish allergies, soy allergies or wheat allergies.

Unwanted immunogenicity can also occur in response to therapeutics. Thus, in another particular embodiment, the disorder is a drug hypersensitivity. “Drug hypersensitivity” refers to an immune-mediated reaction to a drug. Symptoms range from mild to severe and include rash, anaphylaxis, and serum sickness. The drug causing the hypersensitivity is not particularly limited in the sense of the present disclosure.

Increasing efforts are being made to reduce immunogenicity to life saving gene therapy vectors. Adverse immune responses to gene therapy vectors can often cause severe toxicities, as well as the formation of neutralizing antibodies that may prevent any needed redosing. Thus, in one embodiment, the liposomal composition is for use in preventing or treating adverse effects triggered by gene therapy. In another particular embodiment, the liposomal composition is for use in preventing or treating adverse effects triggered by viral vectors, in particular viral transfer vectors. The present disclosure also contemplates the liposomal composition disclosed herein for use in preventing or treating adverse effects triggered by any drug, such as a therapeutic protein.

In another particular embodiment, the disorder is a transplant rejection. “Transplant rejection” occurs when transplanted tissue is rejected by the recipient's immune system, which destroys the transplanted tissue. “transplantation” and variations thereof refers to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). The term “allograft” or “allogenic transplant” or “allotransplant” is the transplanted tissue, for example, an organ, from a donor that is of different genetic origins but of the same species as the recipient. For example, the allograft may be a solid organ, in particular, a kidney, but also a lung, heart, pancreas, liver, etc. The allograft may be any other kind of tissue, such as skin, bone, muscle, vascular tissue, cartilage, etc. As would be evident to the skilled person, encompassed allografts may also be organ tissue, such as renal tissue, lung tissue, heart tissue, liver tissue, etc. As used herein, the term “transplant rejection” or “graft rejection” encompasses both acute and chronic transplant rejection and refers to rejection of the transplanted tissue by the immune system of the recipient. Any type of transplant rejection, to any type of graft, is contemplated in the present disclosure. In one particular embodiment, the immune disorder is graft versus host disease

The particular dose of liposome composition administered according to this disclosure may be determined by the particular circumstances surrounding the case, including the antigen administered, the route of administration, the particular condition being treated, and the similar considerations. Further, the clinical stage of the immune disorder being treated might also need to be taken into account for determining an appropriate dose of the liposome composition to be administered.

Altogether, the dose of liposome composition to be administered is determined in view of several circumstances. In some embodiments, the dose is calculated in terms of the amount of liposomes per Kg of body weight (mg liposomes/Kg body weight). In one embodiment, the dose is in the range from 0.025 to 50 mg of liposomes/Kg of body weight, in particular from 0.25 to 10 mg of liposomes/Kg of body weight.

Further, the medical expert will determine how many doses of the medicament are administered to the subject in need thereof in order to treat the immune disorder. In this respect, it is remarkable that the liposome composition therapy developed by the inventors is not required permanently. Instead, long-lasting restoration of tolerance is achieved by administering the liposome composition of the disclosure, which results in effective treatment of the immune disease. This is in sharp contrast to known immunomodulatory or anti-inflammatory treatments for immune disorders, which are generally life-long treatments. A long-lasting tolerogenic effect can be achieved after a single administration, or alternatively within 2-4 administrations, of the liposome composition of the disclosure. However, the medical expert may decide that more doses are needed to treat advanced stages of the disease, or any other reason. In one embodiment, the treatment comprises administering 1 to 10 doses, for example 2, 3, 4, 5, 6, 7, or 8 doses, of the liposome composition to the subject.

In one embodiment, the subject is in need of antigen-specific tolerance. In another embodiment, the subject has an autoimmune disease, an inflammatory disease, an allergy, graft versus host disease, organ or tissue rejection or has undergone or will undergo transplantation. In another embodiment, the subject has received, is receiving or will receive a therapeutic protein against which they have experienced, are experiencing or are expected to experience an undesired immune response.

As already mentioned above, the present disclosure does not exclude combining the liposome composition described herein with other active agents. Thus, in one embodiment, the liposome composition for any of the uses defined above is for use in combination with a further active agent. The liposome composition and the further active agent may be administered in the same composition or in different compositions and, in the latter case, they can be administered sequentially, simultaneously or within a therapeutic interval. In a particular embodiment, the further active agent is another immunomodulatory agent, for example an immunosuppressant.

For completeness, the present description is also disclosed in the following numbered embodiments:

1. A composition comprising two populations of liposomes, wherein:
the first population of liposomes has a size of or below 200 nm,
the second population of liposomes has a size of or above 500 nm,
the liposomes of the first and second populations carry one or more antigens, and
the liposomal membrane of each liposome in the first and second liposome populations comprises phosphatidylserine in an amount ranging from 20 to 60% by weight with respect to the total composition of the liposome's membrane.

2. A composition consisting essentially of two populations of liposomes, wherein:
the first population of liposomes has a size of or below 200 nm,
the second population of liposomes has a size of or above 500 nm,
the liposomes of the first and second populations carry one or more antigens, and
the liposomal membrane of each liposome in the first and second liposome populations comprises phosphatidylserine in an amount ranging from 20 to 60% by weight with respect to the total composition of the liposome's membrane.

3. A composition according to any one of the preceding embodiments, wherein:
the first population of liposomes has a size in the range from 2 to 200 nm, and
the second population of liposomes has a size in the range from 500 to 2000 nm.

4. The composition according to any one of the preceding embodiments, wherein 15-75% of the liposomes in the composition correspond to the first population.

5. The composition according to the preceding embodiment, wherein 15-55% of the liposomes in the composition correspond to the first population.

6. The composition according to the preceding embodiment, wherein 20-50% of the liposomes in the composition correspond to the first population.

7. The composition according to any one of the preceding embodiments, wherein 2-40% of the liposomes in the composition correspond to the second population.

8. The composition according to the preceding embodiment, wherein 2-25% of the liposomes in the composition correspond to the second population.

9. The composition according to the preceding embodiment, wherein 2-15% of the liposomes in the composition correspond to the second population.

10. The composition according to any one of the preceding embodiments, wherein the amount of phosphatidylserine in the liposome's membrane is from 35 to 45% by weight with respect to the total composition of the liposome's membrane.

11. The composition according to the preceding embodiment, wherein the amount of phosphatidylserine in the liposome's membrane is from 35 to 45% by weight with respect to the total composition of the liposome's membrane, for example 36%, 37%, 38%, 39%, 40%, 41%, 42%, 43%, or 44% by weight with respect to the total composition of the liposome's membrane.

12. The composition according to any one of the preceding embodiments, wherein the PS is selected from the group consisting of 1,2-dioleoyl-phosphatidylserine, 1,2-dioleoyl-sn-glycero3-phospho-L-serine (DOPS), 1,2-dipalmitoyl-phosphatidylserine, 1,2-dimyristoyl-phosphatidylserine, 1,2-distearoyl-phosphatidylserine, I-oleoyl-2-palmitoyl-phosphatidylserine, I-oleoyl-2-stearoyl-phosphatidylserine, 1-palmitoyl-2-oleoyl-phosphatidylserine, I-stearoyl-2-oleoyl-phosphatidylserine, and combinations thereof.

13. The composition according to any one of the preceding embodiments, wherein the liposomal membrane further comprises phosphatidylcholine (PC).

14. The composition according to the preceding embodiment, wherein the amount of PC in the liposome's membrane is from 20 to 50% by weight with respect to the total composition of the liposome's membrane.

15. The composition according to the preceding embodiment, wherein the amount of phosphatidylserine in the liposome's membrane is from 32 to 42% by weight with respect to the total composition of the liposome's membrane, for example 33%, 34%, 35%, 36%, 37%, 38%, 39%, 40%, or 41% by weight with respect to the total composition of the liposome's membrane.

16. The composition according to any one of embodiments 13-15, wherein the PC is selected from the group consisting of 1,2-Dimyristoyl-sn-glycero-3-phosphocholine (DMPC), dipalmitoyl phosphatidylcholine (DPPC), dioleoylphosphatidylcholine, 1,2-dilinoleoyl-sn-glycero-3-phosphocholine, 1-stearoyl-2-linoleoyl-sn-glycero-3-phosphocholine, 1-stearoyl-2-oleoyl-sn-glycero-3-phosphocholine, and combinations thereof.

17. The composition according to any one of the preceding embodiments, wherein the liposomal membrane further comprises sterol lipids or steroids, in particular, cholesterol (CHOL).

18. The composition according to the preceding embodiment, wherein the amount of CHOL in the liposome's membrane is from 10 to 40% by weight with respect to the total composition of the liposome's membrane.

19. The composition according to the preceding embodiment, wherein the amount of CHOL in the liposome's membrane is from 20 to 35% by weight with respect to the total composition of the liposome's membrane, or from 20 to 30% by weight with respect to the total composition of the liposome's membrane, for example 21%, 22%, 23%, 24%, 25%, 26%, 27%, 28%, or 29% by weight with respect to the total composition of the liposome's membrane.

20. The composition according to any one of the preceding embodiments, wherein the liposome's membrane comprises PS, PC and CHOL.

21. The composition according to the preceding embodiment, wherein the liposome's membrane consists essentially of PS, PC and CHOL.

22. The composition according to any one of embodiments 1-21, wherein the molar ratio PS:PC:CHOL is 1:(0.2-4):(0.2-5).

23. The composition according to the preceding embodiment, wherein the molar ratio PS:PC:CHOL is 1:(0.6-1.8):(0.7-2.5), in particular 1:(0.7-1.5):(0.9-2).

24. The composition according to the preceding embodiment, wherein the molar ratio PS:PC:CHOL is 1:(0.8-1.4):(1.1-1.9), in particular, 1:(0.9-1.3):(1.2-1.7).

25. The composition according to any one of the preceding embodiments, wherein the liposomes of the first population provide 5-30% by weight of the total liposomal PS in the composition, in particular 5-20% by weight of the total liposomal PS in the composition.

26. The composition according to any one of the preceding embodiments, wherein the liposomes in of the second population provide 5-75% of the total liposomal PS in the composition, in particular 5-55% by weight of the total liposomal PS in the composition.

27. The composition according to any one of the preceding embodiments, wherein liposomes contain one type of antigen.

28. The composition according to any one of the preceding embodiments, wherein liposomes contain more than one type of antigen.

29. The composition according to any one of the preceding embodiments, wherein the antigen is a peptide.

30. The composition according to the preceding embodiment, wherein the antigenic peptide has a size from 5 to 1000 amino acids.

31. The composition according to the preceding embodiment, wherein the antigenic peptide has a size from 5 to 200 amino acids, for example from 15 to 100 amino acids.

32. The composition according to any one of the preceding embodiments, wherein the antigen is a self-antigen.

33. The composition according to the preceding embodiment, wherein the self-antigen is associate with an autoimmune disease selected from the group consisting of associated to type 1 diabetes (T1D), lupus erythematosus, rheumatoid arthritis, juvenile rheumatoid arthritis, osteoarthritis, reactive arthritis, psoriatic arthritis, multiple sclerosis, neuromyelitis optica, Addison's disease, celiac disease, dermatomyositis, Hashimoto's thyroiditis, myasthenia gravis, pernicious anemia, autoimmune hemolitic anemia, autoimmune neutrophenia, Graves' disease, psoriasis, Sjogren syndrome, including keratoconjunctivitis sicca secondary to Sjogren's Syndrome, autoimmune thyroiditis, dermatitis (including atopic dermatitis and eczematous dermatitis), alopecia areata, Crohn's disease, aphthous ulcer, iritis, conjunctivitis, keratoconjunctivitis, ulcerative colitis, autoimmune uveitis, allergic encephalomyelitis, acute necrotizing hemorrhagic encephalopathy, idiopathic bilateral progressive sensorineural hearing loss, aplastic anemia, pure red cellanemia, idiopathic thrombocytopenia, immune-mediated thrombotic thrombocytopenia purpura, polychondritis, Wegener's granulomatosis, chronic active pepatitis, Stevens Johnson syndrome, idiopathic sprue, lichen planus, sarcoidosis, primary biliary cirrhosis, uveitis posterior, and interstitial lung fibrosis.

34. The composition according to any one of embodiments 32-33, wherein the self-antigen is selected from the group consisting of insulin, proinsulin, protein tyrosine phosphatase (IA2), glutamate decarboxylase (GAD), chromogranin and islet-glucose-6-phosphatase catalytic subunit-related protein (IGRP), peripherin, myelin, myelin-oligodendrocyte glycoprotein (MOG), myelin basic protein (MBP), myelin proteolipid protein (PLP), GDP-I-fucose synthase, acetylcholine receptor (AChR), Muscle-specific tyrosine kinase (MuSK), Agrin, lipoprotein related protein 4 (LRP4), cortactin, transglutaminase, deamidated gliadin, thyroglobulin, collagen (e.g., collagen type 11), human cartilage gp 39, chromogranin A, gp130-RAPS, vimentin, citrullinated vimentin, ADAMST13, aquaporin-4, proteolipid protein, fibrillarin, nuclear proteins, nucleolar proteins (e.g., small nucleolar protein), histidyl-tRNA synthetase (HisRS), histidine-tRNA synthetase (HARS1), jo-1, thyroid stimulating factor receptor, histones, glycoprotein gp 70, ribosomal proteins, pyruvate dehydrogenase dehydrolipoamide acetyltransferase, hair follicle antigens, human tropomyosin isoform 5, mitochondrial proteins, pancreatic β-cell proteins, gluten, and antigenic fragments or derivatives of any of the above, in particular the self-antigen is selected from the group consisting of MOG, PLP, MBP, Pre-proinsulin, insulin, peripherin, acetylcholine receptor, MUSK, citrullinated vimentin, collagen, deamidated gliadin, ADAMST13, Aquaporin 4, transglutaminase, histidyl-tRNA synthetase (HisRS), and antigenic fragments or derivatives thereof, for example, an antigenic fragment selected from those disclosed in table 3.

35. The composition according to any one of embodiments 32-33, wherein the self-antigen is selected from one of the antigens disclosed in table 1, one of the antigens disclosed in table 2, and antigenic fragments or derivatives of the antigens of table 1 or 2.

36. The composition according to any one of embodiments 1-31, wherein the antigen is a drug, including therapeutic protein.

37. The composition according to any one of embodiments 1-31, wherein the antigen is an allergen.

38. The composition according to any one of embodiments 1-31, wherein the antigen is an alloantigen.

39. The composition according to any one of embodiments 1-31, wherein the antigen is a viral antigen, in particular, a viral vector, more in particular the antigen is a viral envelope protein or a viral capsid protein, for example selected from VP1, VP2 or VP3.

40. The composition according to any one of embodiments 36-39, wherein the antigen is a protein fragment, preferably an immunogenic fragment.

41. The composition according to any one of the preceding embodiments, wherein the antigen is a polynucleotide encoding for the antigenic protein or fragment thereof.

42. The composition according to any one of the preceding embodiments, wherein the composition does not contain an immunosuppressant.

43. A method for preparing a liposome composition as defined in any one of the preceding embodiments, said method comprising the steps of:
(a) preparing a lipid blend in an appropriate solvent, and
(b) injecting said lipid blend in a solution containing at least one antigen and an appropriate buffer.

44. The method according to embodiment 43, wherein the lipid blend comprises PS, PC and CHOL.

45. The method according to any one of embodiments 43-44, wherein the molar ratio PS:PC:CHOL is in the range from 1:(0.8-1.4):(1.1-1.9), in particular in the range from 1:(0.9-1.3):(1.2-1.7).

46. The method according to any one of embodiments 43-45, wherein the solvent in step (a) is ethanol.

47. The method according to any one of embodiments 43-46, wherein the buffer in step (b) is phosphate buffer saline.

48. The method according to any one of embodiments 43-47, wherein step (b) comprises slow speed injection and gentle shaking.

49. The method according to any one of embodiments 43-48, further comprising a separation step selected from filtration, e.g., tangential flow filtration, centrifugation (e.g., ultracentrifugation), size extrusion chromatography, gel-permeation chromatography, and combinations thereof.

50. A liposome composition obtainable by a method as defined in any one of embodiments 43-49.

51. The composition according to any one of embodiments 1-42 or 50, that is a pharmaceutical composition and comprises pharmaceutically acceptable excipients and carriers.

52. The composition according to the preceding embodiment, that is for intravenous, intraarterial, intraperitoneal (i.p.), subcutaneous, intramuscular or intradermal administration.

53. The composition according to any one of embodiments 51-52, further comprising an additional active ingredient.

54. A kit of parts that comprises:
(a) a liposome composition according to any one of embodiments 1-42 or 50-53;
(b) optionally, a further active ingredient; and
(c) optionally, instructions for its use.

55. A composition as defined in any one of embodiments 1-42 or 50-53, for use as a medicament.

56. A composition as defined in any one of embodiments 1-42 or 50-53, for use in inducing tolerance to an antigen.

57. The composition for use according to embodiment 56, wherein the inducing of tolerance comprises B-cell mediated tolerance and T-cell mediated tolerance to the antigen.

58. A composition as defined in any one of embodiments 1-42 or 50-53, for use in immunomodulation.

59. A composition as defined in any one of embodiments 1-42 or 50-53, for use in suppressing an excessive immune response.

60. A composition as defined in any one of embodiments 1-42 or 50-53, for use in in the treatment of a disorder associated to an abnormal immune response, wherein said liposome composition restores tolerance to the antigen comprised in the liposome composition.

61. A composition as defined in any one of embodiments 1-42 or 50-53, for use in the treatment of an autoimmune disease.

62. The composition for use according to the preceding embodiment, wherein the autoimmune disease selected from the group consisting of T1D, lupus erythematosus, arthritis (including rheumatoid arthritis, juvenile rheumatoid arthritis, osteoarthritis, reactive arthritis, psoriatic arthritis), multiple sclerosis, neuromyelitis optica, Addison's disease, celiac disease, dermatomyositis, Hashimoto's thyroiditis, myasthenia gravis, pernicious anemia, autoimmune hemolitic anemia, autoimmune neutrophenia, Graves' disease, psoriasis, Sjogren syndrome, including keratoconjunctivitis sicca secondary to Sjogren's Syndrome, autoimmune thyroiditis, dermatitis (including atopic dermatitis and eczematous dermatitis), alopecia areata, Crohn's disease, aphthous ulcer, iritis, conjunctivitis, keratoconjunctivitis, ulcerative colitis, autoimmune uveitis, allergic encephalomyelitis, acute necrotizing hemorrhagic encephalopathy, idiopathic bilateral progressive sensorineural hearing loss, aplastic anemia, pure red cellanemia, idiopathic thrombocytopenia, immune-mediated thrombotic thrombocytopenia purpura, polychondritis, Wegener's granulomatosis, chronic active pepatitis, Stevens Johnson syndrome, idiopathic sprue, lichen planus, sarcoidosis, primary biliary cirrhosis, uveitis posterior, and interstitial lung fibrosis.

63. The composition for use according to the preceding embodiment, wherein the autoimmune disease selected from the group consisting of selected from the group consisting of T1D, multiple sclerosis, rheumatoid arthritis, neuromyelitis optica, myasthenia gravis, myositis or anti-synthetase syndrome, thrombotic thrombocytopenia purpura, and celiac disease.

64. A composition as defined in any one of embodiments 1-42 or 50-53, for use in the treatment of an allergy.

65. A composition as defined in any one of embodiments 1-42 or 50-53, for use in the treatment of an hypersensitivity reaction, for example, a drug hypersensitivity.

66. A composition as defined in any one of embodiments 1-42 or 50-53, for use in the treatment of a transplant rejection.

67. A composition as defined in the preceding embodiment, wherein the transplant rejection is graft versus host disease.

68. A composition as defined in any one of embodiments 1-42 or 50-53, for use in the treatment of an adverse immune effect triggered by gene therapy.

69. The composition according to any one of embodiments 1-42 or 50-53, for use in combination with a further active ingredient.

70. The composition for combined use according to the preceding embodiment, wherein the composition as defined in any one of embodiments embodiments 1-42 or 50-53 and the further active ingredient are administered sequentially, simultaneously or within a therapeutic interval.

71. The composition for combined use according to any one of embodiments 69-70, wherein the further active ingredient is not an immunosuppressant.

72. The composition for use according to any one of embodiments 55-71, wherein the dose of the liposome composition is in the range from 0.25 to 50 mg liposomes/Kg body weight.

73. The composition for use according to any one of embodiments 60-72, wherein the treatment is prophylactic treatment.

Throughout the description and claims the word “comprise” and variations of the word, are not intended to exclude other technical features, additives, components, or steps. Furthermore, the word “comprise” encompasses the case of “consisting of” and “consisting essentially of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### 1. Materials & Methods

### 1.1. Liposome Manufacturing

Antigenic peptides (antigens) were selected based on their reported relevance in autoimmune disease development. The peptides—human mutated citrullinated Vimentin (MCV) (SEQ ID NO: 44), MOG₃₅₋₅₅ (SEQ ID NO:4) and human AChR₁₄₆₋₁₆₂ (SEQ ID NO: 57)—were purchased from Peptide Synthesis Facility (Department of Experimental and Health Sciences, Pompeu Fabra University, Spain) with >95% purity; human insulin (CAS reference number: 11061-68-0; Ins(h)) was purchased from Sigma Aldrich (USA). They were resuspended with Phosphate Buffered Saline (DPBS, Fisher Scientific, Spain) to 0.5 mg/ml. Liposomes were manufactured in Ahead Therapeutic's cleanroom (Arboç, Spain) under GMP-like conditions.

Liposomes were composed of 1,2-dioleoyl-sn-glycero3-phospho-L-serine (sodium salt) (DOPS, Lipoid GmbH, Germany), 1,2-Dimyristoyl-sn-glycero-3-phosphocholine (DMPC, Lipoid), and cholesterol (CH, Sigma Aldrich).

Liposomes were prepared by solvent injection method. DMPC, DOPS and cholesterol were dissolved in Ethanol (EtOH, Sigma Aldrich) and rapidly injected into the DPBS solution containing the required antigen, having a final lipid concentration of 30 mM. Liposome emulsion was twice extruded through a polycarbonate membrane of 1 μm pore size using a Lipex Thermobarrel Extruder (Evonik, Canada) and sonicated during 10 minutes in an ultrasonic bath sonicator (Bandelin, Sonorex RK100) to obtain the liposomal compositions of the invention (abbreviated as PS-PC-Chol-Liposomes-200&500).

PS-PC-Chol-Liposomes under 200 nm (abbreviated as PS-PC-Chol-Liposomes-<200) were obtained by multiple extrusion through 200 nm or 100 nm pore size membranes.

PS-PC-Chol-Liposomes with a diameter above 500 nm (abbreviated as PS-PC-Chol-Liposomes->500) were prepared as described in WO2015107140A1.

Fluorescent liposomes were prepared with either 3-hexanoyl-Nitrobenzoxadiazole Cholesterol (NBD, Cayman Chemical, USA) or Alexa Fluor 750 dye (ThermoFisher Scientific, USA). DOPE-AF750 dye was obtained by incubation of 0.11 μL of TEA and 4 mg of 1,2-Dioleoyl-sn-glycerophosphoethanolamine (DOPE) in a solution of 0.5 mg/ml Alexa Fluor 750 in Ethanol. The solution was stirred for 5 h. 0.15% molar of either 3-hexanoyl-NBD Cholesterol or DOPE-AF750 dye was added to the Ethanol solution of DOPS, DMPC, and cholesterol before the injection to DPBS solution.

The particle size was measured using Nano Tracking Analysis (NTA) using a Malvern Panalytical Nanosight NS300, and ζ-potential was measured by dynamic light scattering using a Zetasizer Nano ZS (Malvern, Instruments Ltd, UK). Peptide encapsulation efficiencies (EE) were calculated indirectly via the equation EE (%)=[(total peptide−free peptide)/total peptide]×100, where total peptide was the total concentration of peptide in the formulation, and the free peptide was the concentration of non-encapsulated peptide. To measure the free peptide, liposome emulsions were centrifuged at 3700 g at room temperature for 20 min and the concentration of non-encapsulated peptide was assessed in the supernatants by PIERCE BCA protein assay kit (Thermo Fisher Scientific Inc., USA).

The unencapsulated peptide was removed from the liposome suspension using 100 KDa Spectra/Por 7 dialysis Tube (SPECTRUM; 131420). Suspension was dialyzed against 50-60 volumes of DPBS Ca/Mg with mild stirring for 24 hours at room temperature, changing once the dialysis bath.

### 1.2. Mice

Wild-type non-obese diabetic (NOD) mice were purchased from the Jackson Laboratory (USA) and bred in the Centre for Comparative Medicine and Bioimage (Badalona, Spain) under specific-pathogen-free (SPF) conditions. This mouse strain develops autoimmune diabetes spontaneously after 12 weeks of age. C57BL/6J 8-week-old female mice purchased from Envigo Laboratories (Milan, Italy) were used for experimental autoimmune encephalomyelitis (EAE) experiments.

All mice were housed in a temperature- and humidity-controlled facility with free access to food and water and subjected to a 12-hour dark/light cycle. All experiments with animal models were performed according to the rules and regulations of the animal facilities, in full compliance with the recommendations stated in the Declaration of Helsinki for animal experimental investigation and in the Principles of Laboratory Animal Care by the National Institute of Health. The protocols received the approval of the animal ethical committee of the governments of the institutions participating in the study.

### 1.3. Liposome Interaction With Murine B Cells (Splenocytes)

The spleen of 12-weeks non-obese diabetic (NOD) mice (Jackson Laboratory, Bar Harbor, ME, USA) was harvested and mechanically disrupted. The cell suspension was hemolyzed and washed twice prior to cell counting by flow cytometry using 7aad (BD Biosciences, San Jose, CA, USA). Afterwards, 10⁶ cells/ml of splenocytes were cultured in RPMI-1640 media (Biowest, Nuaillé, France) plus 10% Fetal Bovine Serum (ThermoFisher Scientific, Waltham, MA, USA), 100 IU/ml penicillin (Normon SA, Madrid, Spain) and 100 μg/ml streptomycin (Laboratorio Reig Jofré, Sant Joan Despí, Spain), and incubated for 4 h at 37° C. and 5% CO₂ without stimulus, with 1 mM of PS-PC-Chol-Insh-liposomes-<200 (non-fluorescent) or with 1 mM PS-PC-Chol-NBD-Insh-liposomes-<200 (fluorescent). After incubation, cells were harvested, washed and stained with 7aad, B220 BV510, CD19 APC-R700 (BD Biosciences) and CD1d PECy7 (BioLegend, San Diego, CA) for 20 min at 4° C. After washing, cells and positivity for NBD were analyzed by flow cytometry using a FACS Fortessa (BD Biosciences), and data were analyzed with FlowJo software (Tree Star, Ashland, OR, USA). The experiment was performed three times in duplicates.

### 1.4. EAE Induction and Clinical Follow-Up

Anesthetized mice were immunized by subcutaneous injections of 100 μl phosphate-buffered saline (PBS) containing 100 μg mouse MOG peptide 35-55 (MOG₃₅₋₅₅) (Proteomics Section, Universitat Pompeu Fabra, Barcelona, Spain) emulsified in 100 μl Complete Freund's Adjuvant (Sigma Chemicals) containing 4 mg/ml Mycobacterium tuberculosis H37RA (Difco Laboratories, Franklin Lakes, NJ, USA). At days 0 and 2 post immunization (p.i.), mice were intravenously injected with 250 ng pertussis toxin (Sigma Chemicals). Mice were weighed and examined daily for neurological signs using the following criteria: 0=no clinical signs; 0.5=partial loss of tail tonus for 2 consecutive days; 1=paralysis of whole tail; 2=mild paraparesis of one or both hindlimbs; 2.5=severe paraparesis or paraplegia; 3=mild tetraparesis; 4=tetraparesis (severe in hindlimbs); 4.5=severe tetraparesis 5=tetraplegia; 6=death (Gutierrez et al., Mol Neurobiol. 2017). All data presented are in accordance with the guidelines suggested for EAE publication (Baker D. et al., J.Neuroimmunol. 2012). Weight loss was calculated as the percentage change in daily weight compared with the initial weight on the day of immunization. Score 5 and weight loss>30% were defined as endpoint criteria to minimize suffering and guarantee animal welfare. Clinical score was monitored for 28 days.

### 1.5. Liposome Biodistribution in NOD Mice

For the in vivo liposome tracking, near-infrared (NIR, 0.7-1.7 μm) fluorescence imaging was performed using the Pearl Impulse imaging system (LI-COR, USA). Prediabetic (<12 weeks-of-age) NOD mice were treated with empty fluorescent DOPE-AF750-PS-liposomes administered via intravenous (i.v.) route in a single dose of 100 μl. In vivo imaging was performed at 1 h, 6 h and 24 h after the injection. Axillary lymph node (LN), bladder, brain, heart, hind limb's bones, kidneys, liver, lungs, mediastinal LN (MDLN), pancreas, perigonadal adipose tissue (PAT), pancreatic LN (PLN), salivary glands, spleen, stomach, and thymus were harvested, washed with DPBS, and finally imaged ex vivo with Pearl Impulse system (LI-COR). Background fluorescence was subtracted in order to normalise fluorescence values. The fluorescence signal was expressed as relative fluorescence units (RFU) per grams of tissue of each organ.

### 1.6. In Vivo Capture of Fluorescent Liposomes in NOD Mice

With the aim to characterize in vivo liposome interactions with B cells, Dendritic cells and other APCs, such as, macrophages and LSECs, prediabetic NOD mice were treated with fluorescent-labelled empty NBD-PS-liposomes or NBD-PSIns(h)-liposomes administered i.v. in a single dose of 100 μl. Spleen was collected at 1 h and 6 h post-injection. Splenocytes, obtained after mechanical disruption and erythrocyte lysis, were then labelled using monoclonal antibodies for 20 min at 4° C. The antibody panel used for this experiment consisted in CD11c BV786, B220/CD45R BV510, CD205/DEC-205 BV421, CD8a PECy7, MHC-II/MHC-I-A[d] PE, CD19 APCCy7. Fixable Viability Stain 575V (BD Biosciences, USA) staining was used to exclude dead cells. Cells were acquired using flow cytometry (FACS LSR Fortessa, BD Biosciences) and corresponding fluorescence minus one (FMO) staining was used as a control. FlowJo software (Tree Star, OR, USA) was used for the data analysis.

TABLE 4
Immune cell subsets determined by flow cytometry analysis

| Subset name | Phenotype |
|---|---|
| B cell | CD19⁺ |
| CD1d^{high}CD5⁺ B cell | CD19⁺CD1d^{high}CD5⁺ |
| B1a B cell | CD19⁺CD5⁺CD43⁺ |
| B1b B cell | CD19⁺CD5⁻CD43⁺ |
| Marginal Zone (MZ) B cell | CD19⁺CD21^{high}CD23⁻ |
| Foxp3⁺ B cell | CD19⁺Foxp3⁺ |
| CD1d⁺LAG3⁺ B cell | CD19⁺CD1d⁺LAG3⁺ |
| CDC (cDC) | CD11c⁺MHC-II⁺CD205⁺CD8a⁺ |
| pDCs | CD11clowMHCII+B220+ |
| Tcells | CD45+CD3+ |
| Macrophages | F4/80+ |
| Kuppfer cells | F4/80+CD68+ |
| LSECs | CD206+F4/80− |

DC, dendritic cell.

### 1.7. In Vitro Liposome Capturing Assessment by Flow Cytometry in NOD's Splenocytes

Splenocytes from female NOD mice were obtained after mechanical disruption and erythrocyte lysis. Cells were then cultured at 3×10⁵ cells/well, in 96-well round-bottom plates, in 200 μl RPMI 1640 (Lonza, Switzerland) supplemented with 10% heat-inactivated foetal bovine serum (Gibco, USA), 2 mM L-glutamine (Corning, USA), 1 mM sodium pyruvate (Lonza), 50 μM 2β-mercaptoethanol (Sigma Aldrich), 100 U potassium penicillin (Lonza) and 100 μg Streptomycin Sulphate (Lonza). They were incubated with 1 mM NBD-PS-liposomes or NBD-PSIns(h)-liposomes for 2 h at 37° C. and 5% CO₂ and maintained overnight. IL-10⁺ cells were stained using the APC Mouse IL-10 Secretion Assay Detection Kit (Miltenyi Biotech, Germany) following the manufacturer's instructions, and then cells were re-incubated with 1 mM NBD-PS-liposomes or NBD-PSIns(h)-liposomes for 1 h to reinforce NBD marking. B cell subsets were identified by monoclonal antibody immunophenotyping, with panels designed as follows: (1) CD19 BV510, CD5 BV421, CD1d PECy7, CD43 PerCP-Cy5.5, LAG3 APC; (2) CD19 BV510, CD21 APCCy7, CD23 BV421. Cells were acquired using flow cytometry (FACS LSR Fortessa, BD Biosciences) and corresponding FMO staining was used as a control. FlowJo software (Tree Star) was used for the data analysis.

### 1.8. IL-10 and TGF-β Analysis in PBMCs

Twenty milliliters of peripheral blood from adult healthy blood donors were obtained by venipuncture into heparin tubes (BD Biosciences). Blood was diluted 1:1 with Phosphate Buffered Saline and density gradient centrifugation with Ficoll (GE Healthcare Life Sciences, Marlborough, MA, USA) was performed to obtain peripheral blood mononuclear cells (PBMCs). Cells were washed and resuspended in X-VIVO 15 media (Lonza, Basel, Switzerland) supplemented with 2% male AB human serum (Biowest), 100 IU/ml penicillin (Normon SA, Madrid, Spain) and 100 μg/ml streptomycin (Laboratorio Reig Jofre). After counting and viability assessment with 7aad (BD Biosciences) by flow cytometry, cells were cultured for 24 h at 37° C. and 5% CO₂ with the different liposomal compositions. After incubation, cells were harvested, washed and stained with CD19 BV785, CD14 BV711, CD3 BV650, CD4 BV570, CD8 APCCy7, CD68 BV421 (BioLegend), TGF-β/LAP PE (Miltenyi Biotec), CD11c PECy7 and 7aad (BD Biosciences). IL-10 secretion was analyzed using the IL-10 Secretion Assay-Detection Kit, human (Miltenyi Biotec) following the manufacturer's instructions. The subsets within PBMCs, NBD positivity, IL-10 secretion and TGF-β expression were assessed with FACS Fortessa (BD Biosciences) and data were analyzed with FlowJo software (Tree Star). The experiment was performed three times in duplicates.

### 1.9. Statistical Analysis

Statistical analysis was performed using the Prism 9.0 software (GraphPad Software Inc., San Diego, CA). For comparison between unpaired data, a parametric two-tailed Student's t-test (Gaussian distribution) or non-parametric Mann-Whitney was used. For comparison among multiple groups, one-way ANOVA (Gaussian distribution) or two-way ANOVA with the corresponding multiple comparison test were performed. P-value<0.05 was considered significant.

### 2. Results

### 2.1. PS-PC-Chol Liposomes Under 200 nm Interact With B Cells and Induce IL-10 Secretion

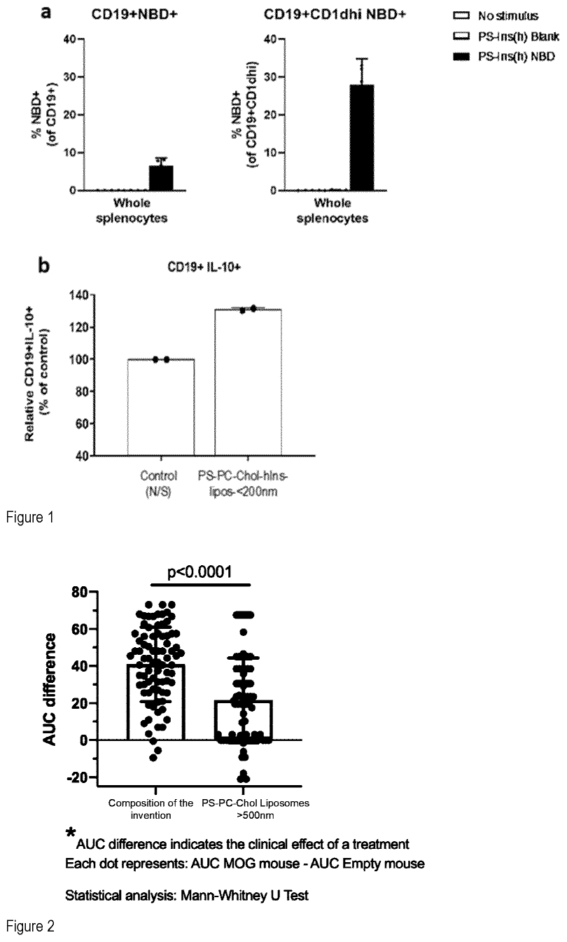

PS-PC-Chol-NBD-Ins(h)-liposomes-<200 were manufactured by Ethanol injection and extruded 10 times with a 200 nm pore membrane, obtaining a composition with 98% of the liposomes with a diameter under 200 nm according to NTA determination, and ζ-potential of −20.0 mV. Those liposomes were incubated with splenocytes for 4 hours and the % of interacting B cells (CD19+) was assessed. Surprisingly we found that 6.2% of CD19+ cells interacted with PS-PC-Chol-NBD-Ins(h)-liposomes-<200 and, even more remarkably, this % increased to 28% when we assessed the interaction in the putative Breg compartment CD19+CD1dhi (FIG. 1).

To determine whether this interaction had a biological significance in terms of inducing tolerance, we analyzed the expression of tolerogenic cytokine, IL-10, in B cells from PBMCs samples after incubating them with PS-PC-Chol-Ins(h)-Liposomes-<200 (10-200 nm 97.5%; >500 nm 0%; ζ-potential −20.0 mV) for 24 h. As shown in the figure, PS-PC-Chol-insulin-liposomes-<200 induce ex vivo expression of IL-10 in human B cells, since we observed an increase of 30% in the expression of this cytokine in B cells after the incubation with those liposomes.

### 2.2. PS-PC-Chol-Liposomal Composition of the Invention has Higher Efficacy Than PS-PC-Chol-Liposomes>500 nm

EAE was induced by MOG immunization as described above and mice were treated with 50 μL of PS-PC-Chol-MOG₃₆₋₅₅-liposomes-200&500 (10-200 nm 43.3%; >500 nm 3.9%; ζ-potential −23.9 mV), PS-PC-Chol-MOG₃₆₋₅₅-liposomes->500 (10-200 nm 0%; >500 nm 95.2%; ζ-potential −24.7 mV), PS-PC-Chol-empty-liposomes-200&500 (10-200 nm 26%; >500 nm 8.2%; ζ-potential −22.7 mV), or PS-PC-Chol-empty-liposomes->500 (10-200 nm 0%; >500 nm 93.1%; ζ-potential −23.2 mV) via injection with 1-mL syringes and 30-G needles, using intravenous route of administration at day 5 post immunization and clinical score was monitored for 28 days.

Area Under the Curve (AUC) difference of clinical score was calculated to compare the beneficial effect of both liposomal compositions. The AUC score of each MOG charged liposome-treated mouse was subtracted from the AUC score of each control mouse (Empty group). A positive AUC difference means that the AUC score was higher in the Empty liposome-treated mouse than in the MOG-charged liposome-treated mouse; this is the MOG-charged liposome-treated mouse presented a milder EAE clinical course.

As shown in FIG. 2, PS-PC-Chol-MOG₃₆₋₅₅-liposomes-200&500 display higher efficacy in controlling the disease progression and clinical symptoms than PS-PC-Chol-MOG₃₆₋₅₅-liposomes->500.

### 2.3. Fluorescent-Labeled PS-PC-Chol-Liposomes-200&500 are Distributed to Target Organs and Captured by Phagocytes in NOD Mice

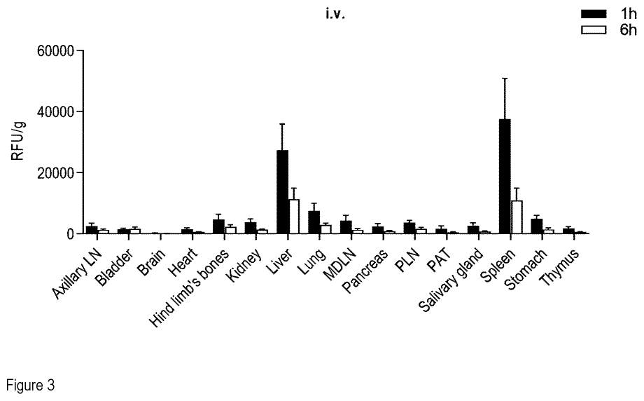

Biodistribution of PS-PC-Chol-liposomes-200&500 was studied using PS-PC-Chol-Empty-liposomes-200&500 labeled with AF750. Thus, PS-PC-Chol-AF750-empty-liposomes-200&500 (10-200 nm 45.1%; >500 nm 4.7%; ζ-potential −24.7 mV) were administered by i.v. route, and fluorescent signal in each organ was evaluated after 1-, 6- and 24 hours post-administration (FIG. 3). i.v. route showed peak values 1 h after injection, and the signal diminished along the time to be negligible by 24 h checkpoint. As shown in the figure, when administering PS-PC-Chol-AF750-empty-liposomes-200&500 i.v., fluorescence specially gathered in the liver, lung, MDLN, spleen and stomach.

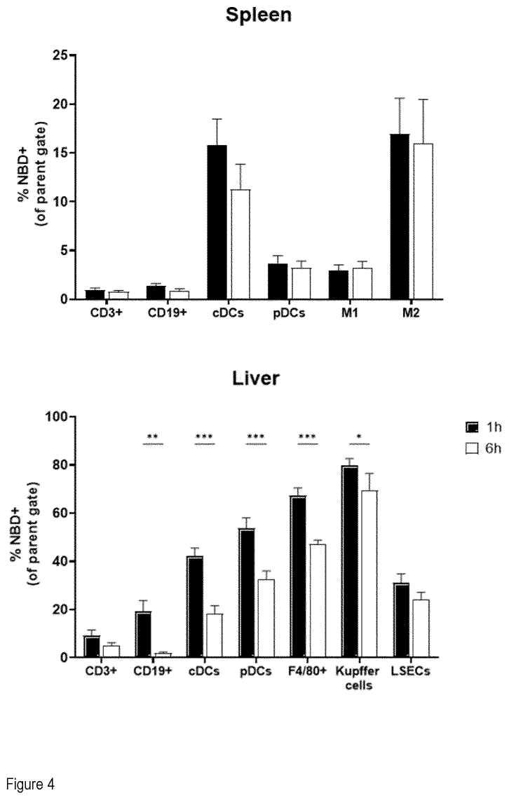

To confirm which immune cells interacted with the composition of the invention in vivo. NOD mice received a single dose i.v of PS-PC-Chol-NBD-insulin-liposomes-200&500 (10-200 nm 37.1%; >500 nm 16.3%; ζ-potential −24.3 mV). Spleens and livers were harvested 1 h and 6 h post-administration. The splenocytes viability was 98.8±0.23 (Mean±SEM, data not shown). In the spleen, conventional DCs (cDCs) (8.6±2.1%) showed NBD signal and the 1.4±0.5% of B cells were also positive for NBD staining, indicating that the formulation of the invention directly interacts with these two immunologic compartments. In the liver, besides the confirmation of the interaction with DCs and B cells, among the immune cells studied, macrophages display the highest % referring to the parent population of interaction with the liposomes. Remarkably, LSECs were also found to be interacting with the liposomes (FIG. 4)

### 2.4. PS-Liposomes Interact With Breg-Cell Subsets and Promote IL-10 Secretion

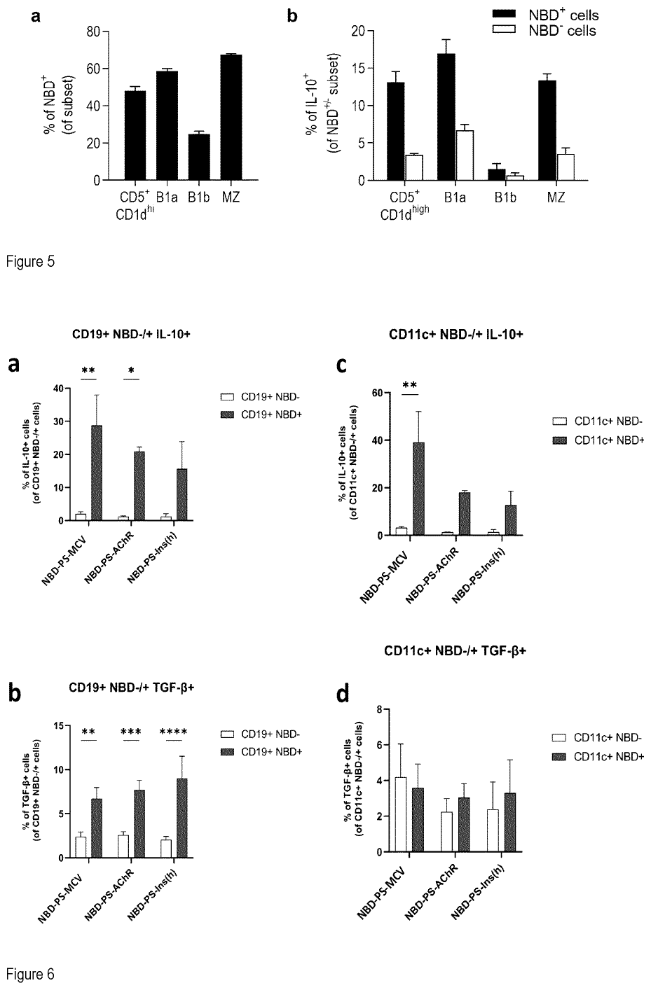

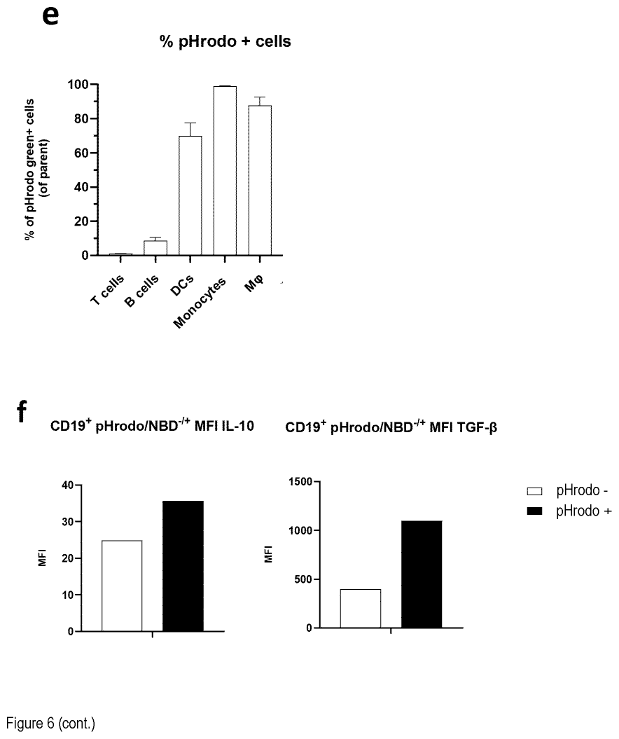

Because B-cells play an important role as antigen-presenting cells (APCs), we confirmed the tolerogenic effect of PS-PC-Chol-Ag-liposomes-200&500 in B-cells. Thus, splenocytes from NOD mice were co-cultured with PS-PC-Chol-NBD-insulin-liposomes-200&500 (10-200 nm 36.4%; >500 nm 18%; ζ-potential −25 mV). Among all B lymphocytes, 34.8±2.0% showed NBD fluorescence. When we dissected NBD fluorescence in the different B-cell subsets with described regulatory potential, CD5⁺CD1d^{high} cells showed 48.1±2.3% of NBD signal (with respect to the whole CD5⁺CD1d^{high} subset); NBD⁺ B1a B lymphocytes, 58.7±1.4% of all B1a B lymphocytes; NBD⁺ B1b B lymphocytes, 24.8±1.5% of all B1b B lymphocytes, and NBD⁺ marginal zone (MZ) B-cells, 67.5±0.4% of all MZ B-cells (FIG. 5). To demonstrate their regulatory function after treatment, positively-secreting IL-10 cells were found in each subset: 13.1±1.4% of CD5⁺CD1d^{high} NBD⁺ cells, 16.9±1.9% of B1a NBD⁺ cells, 1.5±0.8% of B1b NBD⁺ cells, and 13.4±0.9%, of MZ NBD⁺ cells (FIG. 5). IL-10 secretion showed a biological tendency to be higher in NBD-fluorescent B-cells when compared to non-fluorescent B-cells for each subset.

Liposomes of the composition of the invention induce IL-10 and TGF-β secretion in B and Dendritic cells from PBMCs

To confirm the tolerance-induction capacity of the composition of the invention in human cells, PBMCs from healthy donors were incubated for 24 h in with or without 100 ng/ml lipopolysaccharide, 1 mM of PS-PC-Chol-NBD-Ins(h)-liposomes-200&500 (10-200 nm 37.0%; >500 nm 2.8%; ζ-potential −22.3 mV), 1 mM of PS-PC-Chol-NBD-MCV-liposomes-200&500 (10-200 nm 39.3%; >500 nm 3.6%; ζ-potential −24.7 mV), or 1 mM of PS-PC-Chol-NBD-AChR-liposomes-200&500 (10-200 nm 18.1%; >500 nm 13.6%; ζ-potential −24.9 mV), or PS-PC-Chol-MCV-200&500 stained with pHrodo™ green Dye for labeling amines according to manufacturer instructions (10-200 nm 28.6%; >500 nm 9.4%; ζ-potential −23.7 mV), and the expression of the tolerance related cytokines IL-10 and TGF-β, was analysed in NBD positive and negative subsets of B cells and DCs. The expression of the two cytokines was also assessed in the pHrodo positive and negative subsets of the B cells.

As shown in the figure, the interaction with the differently loaded liposomes induces a clear increase in the expression of IL-10 and TGF-β in CD19+ compartment, while DCs capturing liposomes do express higher levels of IL-10 than the ones that do not engulf the liposomes, but no differences are observed in the case of TGF-β expression. Moreover, with the pHrodo staining, which is sensitive to pH and indicates direct engulfment of the cell, if positive, we confirmed that the modulation of the B cells by liposomes treatment was due to the engulfment of the liposomes rather than to membrane indirect interaction. Those data demonstrate that the mechanism of action to induce tolerance through B cells and DCs is common independently of the antigen-loaded in the PS-liposome.

### CITATION LIST

### Patent Literature

WO2015107140

### Non Patent Literature

Chen et al, J Immunol 2020, 204(2): 335-347. https://doi.org/10.4049/jimmunol.1801677
Roep B O, Peakman M. Cold Spring Harb Perspect Med, 2012, vol. 2(4):a007781. doi: 10.1101/cshperspect.a007781
Lernmark A. J. Clin Invest, 2001, vol. 108, p. 1091-1096
Bangham et al., J. Mol. Biol., 13, 238 (1965)
Pons et al. International Journal of Pharmaceutics 95 (1993) 51-56)
Maurer N. et al., Expert Opin Biol Ther, 2001, vol. 1(6), p. 923-47
Waterhouse D. N. et al., Methods Enzymol., 2005; vol. 391, p. 40-57
Urbán P. et al., Nanosc. Res. Lett., 2011, vol. 6, p. 620
Gutierrez et al., Mol Neurobiol. 2017 August;54(6):4820-4831. doi: 10.1007/s12035-016-0154-2.
Baker D. et al., J.Neuroimmunol. 2012 Jan. 18; 242(1-2):78-83. doi: 10.1016/j.jneuroim.2011.11.003
Whitepaper on Nanoscale Material Characterization: a Review of the use of Nanoparticle Tracking Analysis (NTA), 2015 Malvern Instruments Limited

What is claimed is:

1. A composition comprising two populations of liposomes, wherein:
the first population of liposomes has a size in the range from 2 to 200 nm,
the second population of liposomes has a size in the range from 500 to 2000 nm,
the liposomes of the first and second populations carry one or more antigens, and
the liposomal membrane of each liposome in the first and second liposome populations comprises phosphatidylserine in an amount ranging from 20 to 60% by weight with respect to the total composition of the liposome's membrane.

2. The composition according to claim 1, wherein 15-75% of the liposomes in the composition correspond to the first population and 2-40% of the liposomes in the composition correspond to the second population.

3. The composition according to claim 1, wherein the amount of phosphatidylserine in the liposome's membrane is from 35 to 45% by weight with respect to the total composition of the liposome's membrane.

4. The composition according to claim 1, wherein the liposomal membrane further comprises phosphatidylcholine (PC) and cholesterol (CHOL).

5. The composition according to claim 4, wherein the liposomal membrane comprises PS, PC and CHOL in a molar ratio PS:PC:CHOL which is comprised from 1:(0.6-1.8):(0.7-2.5).

6. The composition according to claim 1, wherein the antigen is a peptide having from 5 to 200 amino acids.

7. The composition according to claim 1, wherein the antigen is selected from the group consisting of self-antigens, drugs, including therapeutic proteins, viral vectors, including viral capsid proteins, allergens and alloantigens.

8. The composition according to claim 7, wherein the antigen is a self-antigen associated to an autoimmune disease, in particular the self-antigen is selected from the group consisting of insulin, proinsulin, protein tyrosine phosphatase (IA2), glutamate decarboxylase (GAD), chromogranin and islet-glucose-6-phosphatase catalytic subunit-related protein (IGRP), peripherin, myelin, myelin-oligodendrocyte glycoprotein (MOG), myelin basic protein (MBP), myelin proteolipid protein (PLP), GDP-I-fucose synthase, acetylcholine receptor (AChR), Muscle-specific tyrosine kinase (MuSK), Agrin, lipoprotein related protein 4 (LRP4), cortactin, transglutaminase, deamidated gliadin, thyroglobulin, collagen (e.g., collagen type 11), human cartilage gp 39, chromogranin A, gp130-RAPS, vimentin, citrullinated vimentin, ADAMST13, aquaporin-4, proteolipid protein, fibrillarin, nuclear proteins, nucleolar proteins (e.g., small nucleolar protein), histidyl-tRNA synthetase (HisRS), histidine-tRNA synthetase (HARS1), jo-1, thyroid stimulating factor receptor, histones, glycoprotein gp 70, ribosomal proteins, pyruvate dehydrogenase dehydrolipoamide acetyltransferase, hair follicle antigens, human tropomyosin isoform 5, mitochondrial proteins, pancreatic β-cell proteins, gluten, and antigenic fragments or derivatives of any of the above, and immunogenic fragments or derivatives of any of the above.

9. The composition according to claim 7, wherein the antigen is a viral capsid protein, in particular selected from VP1, VP2 and VP3.

10. The composition according to claim 1, which does not contain an immunosuppressant.

11. The composition according to claim 1, that is a pharmaceutical composition and comprises pharmaceutically acceptable excipients and carriers.

12-15. (canceled)

16. The composition according to claim 1, wherein the size of the liposomes is determined by Nanoparticle Tracking Analysis (NTA).

17. The composition according to claim 1, wherein:
15-75% of the liposomes in the composition correspond to the first population and 2-40% of the liposomes in the composition correspond to the second population;
the amount of phosphatidylserine in the liposome's membrane is from 35 to 45% by weight with respect to the total composition of the liposome's membrane;
the liposomal membrane further comprises phosphatidylcholine (PC) and cholesterol (CHOL); and
the molar ratio PS:PC:CHOL is 1:(0.6-1.8):(0.7-2.5).

18. The composition according to claim 17, wherein the antigen is a peptide having from 5 to 200 amino acids selected from the group consisting of self-antigens, drugs, including therapeutic proteins, viral vectors, including viral capsid proteins, allergens and alloantigens.

19. The composition according to claim 18, which does not contain an immunosuppressant.

20. A method for treating a condition selected from an autoimmune disease, allergy, drug hypersensitivity, transplant rejection, and an adverse immune effect triggered by gene therapy, said method comprising administering a therapeutically effective amount of a composition as defined in claim 19 to a subject in need thereof.
